(19) United States
(12) Patent Application Publication
(10) Pub. No.: US 2021/0169927 A1
(43) Pub. Date: Jun. 10, 2021

(54) METHOD OF IMPROVING MUSCLE AND CONNECTIVE TISSUE WITH SHILAJIT

(71) Applicant: Natreon, Inc., New Brunswick, NJ (US)

(72) Inventor: Sanyasi R. Kalidindi, Monroe, NJ (US)

(73) Assignee: Natreon, Inc., New Brunswick, NJ (US)

(21) Appl. No.: 16/702,819

(22) Filed: Dec. 4, 2019

(51) Int. Cl. A61K 35/02; A61K 31/35; A61K 31/194; A23L 33/16; A23L 33/00; A61P 21/00

(52) U.S. Cl. CPC A61K 35/02; A61K 31/35; A61P 21/00; A23L 33/16; A23L 33/30; A61K 31/194

(57) ABSTRACT

The present invention is directed to methods for improving muscle and connective tissue with Shilajit, including reducing collagen degradation and reducing the loss of strength due to muscle fatigue. The present methods improve muscle building and repair as well as the health of skin, cartilage, connective tissues, muscle, vascular tissues, bones, and teeth, and treat collagen-related disorders such as arthritis and osteoporosis.

### TECHNICAL FIELD

The present invention relates to methods for improving muscle and connective tissue with Shilajit, including reducing collagen degradation and reducing the loss of muscle strength due to muscle fatigue. The present methods improve muscle building and repair as well as the health of skin, cartilage, connective tissues, muscle, vascular tissues, bones, and teeth, and treat collagen-related disorders such as arthritis and osteoporosis.

### BACKGROUND

Collagen is the body's major structural protein, composed of three protein chains wound together in a tight triple helix, This unique structure gives collagen a greater tensile strength than steel. Approximately thirty-three percent of protein in the human body is collagen. This protein supports tissues and organs and connects these structures to bones. Bones are composed of collagen combined with certain elements such as calcium and phosphorus. Collagen plays a key role in providing the structural scaffolding surrounding cells that helps to support cell shape and differentiation. The mesh-like collagen network binds cells together and provides the supportive framework or environment in which cells develop and function, and tissues and bones heal.

Collagen, in the form of elongated fibrils, is mostly found in fibrous tissues such as tendons, ligaments, and skin. Collagen is also abundant in corneas, cartilage, bones, blood vessels, the gut, intervertebral discs and the dentin in teeth. In muscle tissue, collagen serves as a major component of the endomysium. Collagen constitutes one to two percent of muscle tissue, and accounts for six (6) percent of the weight of strong, tendinous muscles. The fibroblast is the most common cell that creates collagen.

### Types of Collagen;

Collagen occurs in many places throughout the body, Over ninety (90) percent of the collagen in the body is type I. So far, twenty-eight (28) types of collagen have been identified and described. The five (5) most common types are:
Collagen skin, tendon, vascular ligature, organs, bone (main component of the organic part of bone);
Collagen cartilage (main component of cartilage);
Collagen reticulate (main component of reticular fibers), commonly found alongside Collagen I;
Collagen IV: forms basal lamina, the epithelium-secreted layer of the basement membrane; and
Collagen V: cell surfaces, hair, and placenta.

### Role of Collagen in the Body

Collagen is important to health because it plays a key role in maintaining the health of skin, connective tissues, tendons, bones, and cartilage,

### Muscles

As discussed throughout this application, in muscle tissue, collagen serves as a major component of the endomysium. Collagen constitutes 1-2% of muscle tissue and 6% of the weight of strong, tendinous muscles. Collagen is part of the Extra-Cellular Matrix, providing support to the structure and function of e.g. skeletal muscle.

### Skin Health

Collagen plays an important role in skin health, Collagen I and Collagen III are formed in human skin in a higher proportion relative to other types of collagen and are maintained in a fixed proportion relative to one another in normal skin tissue. Collagen I constitutes about seventy (70) percent of collagen in the skin, with Collagen III constituting about ten (10) percent of collagen in the skin and Collagens IV, V, VI, and VII each constituting trace amounts of collagen in the skin. Collagen maintains firmness and elasticity of the skin. Collagen, in the form of collagen hydrolysate, keeps skin hydrated. Decreases in the amount of collagen in the body with age result in sag, lines, wrinkles, lack of tension and elasticity, and delay in wound healing processes.

### Wound Healing

Collagen is a key protein in connective tissue and plays an important role in wound healing by repair and formation of scars. Age-related delay in wound healing is caused by impaired synthesis and increased degradation of collagen.

### Bone

About 95% of the organic part of the bone is made of collagen, mainly Collagen I. The combination of hard minerals and flexible collagen makes bone harder than cartilage without being too brittle. A combination of collagen mesh and water forms a strong and slippery pad in the joint that cushions the ends of the bones in the joint during muscle movement.

### Cartilage, Tendons, Ligaments

Collagen, in the form of elongated fibrils, is predominantly found in fibrous tissues such as tendons and ligaments. It is a flexible and stretchy protein that is used by the body to support tissues and thus it plays a vital role in the maintenance of cartilage, tendons, and ligaments. Normal tendon consists of soft and fibrous connective tissue that is composed of densely packed collagen fiber bundles and surrounded by a tendon sheath. Collagen II is the major component in cartilage.

### Dental Tissue

The organic part of dentin and pulp consist of collagen, mainly Collagen I, with small amounts of Collagens III and V. The predominant collagen found in cementum is Collagen I, and in periodontal ligaments, are found Collagens I, III, and XII.

### Basement Membrane

The epithelial basement membrane is composed of Collagens IV and VII.

### Collagen-Related Disorders

Collagen-related disorders most commonly arise from genetic defects or nutritional deficiencies that affect the biosynthesis, assembly, post-translational modification, secretion, or other processes involved in normal collagen production. Various collagen-related disorders are described below:

Osteogenses imperfecta is a dominant autosomal disorder caused by a mutation in Collagen I. Osteogenses imperfecta results in weak bones and irregular connective tissue. Some cases can be mild while severe cases can be lethal. Mild cases are characterized by lowered levels of Collagen I while severe cases are characterized by structural defects in collagen.

Chondrodysplasia is a skeletal disorder believed to be caused by a mutation in Collagen II and is the subject of continuing research efforts.

Ehlers-Danlos Syndrome leads to deformities in connective tissues. Ten different types of this disorder are known, some of which are characterized by the rupturing of arteries and are thus lethal. Each type of the Ehlers-Danlos Syndrome is caused by a different mutation; for instance, type four (4) of this syndrome is caused by a mutation in Collagen III.

Alport Syndrome can be passed on genetically, usually as an X-linked dominant gene, but also as both an autosomal dominant and autosomal recessive gene. Individuals suffering from Alport Syndrome experience kidney and eye problems, and childhood or adolescent loss of hearing.

Osteoporosis is experienced with age rather than inherited genetically and is associated with reduced levels of collagen in the skin and bones. Growth hormone injections are being researched as a possible treatment for osteoporosis in order to counteract loss of collagen.

Knobloch syndrome is caused by a mutation in the Collagen XVIII gene. Patients suffering from Knobloch syndrome present with protrusion of brain tissue and degeneration of the retina. Individuals having one or more family members suffering from Knobloch syndrome are at an increased risk of developing it themselves, as there is a hereditary predisposition.

Arthritis is an inflammation of the joints, causing swelling and pain, and gradually degrades collagen in the joints. Rheumatoid arthritis is an autoimmune disease in which protein-degrading enzymes (matrix metalloproteinases) attack collagen and other proteinaceous components of the joints, along with other destructive agents such as inflammatory cells and cytokines. Degenerative joint disease, synonymous with osteoarthritis, Is the most prevalent of all types of arthritis debilitating canines today. Osteoarthritis is a progressive deterioration of the articular cartilage in the joints, which may cause joint effusion, and bone spurs called osteophytes around the margins of the joints. This type of arthritis may also occur due to excessive uncontrolled inflammation around the joints from soft tissue swelling. There are two types of osteoarthritis, primary and secondary. Primary osteoarthritis is characterized by normal aging, or wearing of the cartilage in the joint. Secondary osteoarthritis is characterized as a result from an underlying cause such as hip dysplasia. Both types lead to the loss of cartilage in the joint and the cartilage producing cells. These observations apply to mammals including canines and humans. The extracellular matrix of cartilage is made up of type II collagen and proteoglycans. The body will continually remodel cartilage to maintain a proper volume, but in osteoarthritis the cartilage degrading enzymes matrix metalloproteinases take over. Chondrocytes respond to the loss of cartilage by propagating type II collagen and proteoglycans and the cartilage becomes thick for several years, but degradation will eventually occur.

### Degradation and Decreased Production of Collagen

With age, collagen degrades, and there is a decrease in the production of collagen. As a result, fine lines and wrinkles appear in the skin. Skin also loses its elasticity and sags. Collagen can be preserved by reducing degradation of existing collagen, in addition to increasing the production of new collagen. Degradation of collagen can be reduced by: (a) protecting the skin from UVA and UVB rays; (b) avoiding excessive exposure to sunlight; (c) having a diet including antioxidants to fight free radicals; (d) ingesting Vitamin C, which accelerates production of new collagen; (e) supplementing with collagen-stimulating peptides; and (f) increasing the ability of the body to produce new collagen.

The Extra-Cellular Matrix (ECM) is essential for the development, maintenance, and regeneration of skeletal muscles. Buck et al., “Cell surface receptors for extracellular matrix molecules” 3 ANNU. REV. CELL BIOL. 179 (1987); Purslow, “The structure and functional significance of variations in the connective tissue within muscle” 133 COMP. BIOCHEM. PHYSIOL. A MOL. INTEGR. PHYSIOL. 947 (2002). The ECM is mainly composed of glycoproteins, collagen, and proteoglycans. The ECM is also involved in the macrostructure of muscle, arranging fibers into bundles, bundles into fascicles, and integrating muscle structure with aponeurosis and tendon. Additionally, the ECM is thought to play a vital role in mechano-transduction, transmitting force laterally from the fiber to the tendon and vice versa. Fomovsky et al., “Contribution of extracellular matrix to the mechanical properties of the heart” 48 J. MOL. CELL CARDIOL. 490 (2010); Purslow et al., “The morphology and mechanical properties of endomysium in series-fibred muscles: variations with muscle length” 15 J. MUSCLE RES. CELL MOTIL. 299 (1994); Street, “Lateral transmission of tension in frog myofibers: a myofibrillar network and transverse cytoskeletal connections are possible transmitters” 114 J. CELL PHYSIOL. 346 (1983). Di Lullo et al., “Mapping the ligand-binding sites and disease-associated mutations on the most abundant protein in the human-type I collagen” J Biol Chem. 2001.

The mechanical strength and elasticity of the ECM are critical to its functional performance—it must be strong enough to sustain the loads of contraction, yet elastic enough to prevent tearing under externally applied strains. Purslow, supra, 133 COMP. BIOCHEM. PHYSIOL. A MOL. INTEGR. PHYSIOL. at 947. These properties change both with age and disease, where chronic alterations to the ECM appear to impair muscle function. Lieber et al., “Inferior mechanical properties of spastic muscle bundles due to hypertrophic but compromised extracellular matrix material” 28 MUSCLE NERVE 464 (2003); Zhou et al., “Targeting fibrosis in Duchenne muscular dystrophy” 69 J. NEUROPATHOL. EXP, NEUROL. 771 (2010). Skeletal muscle shows an enormous plasticity to regulate its functional, structural, and metabolic properties in order to adapt to varying physiological demands, McGee and colleagues detected histone deacetylase (HDAC)-specific inhibition patterns and changes in histone acetylation after a single bout of exercise in human skeletal muscle. McGee et al., “Exercise-induced histone modifications in human skeletal muscle” 587 J. PHYSIOL. 5951 (2009).

Physical exercise essentially contributes to a healthy lifestyle and leads to risk reduction, a better prognosis, and a decrease in side effects of medical treatments for several common diseases, including cancer as well as cardiovascular, metabolic, and neurodegenerative disorders. Lee et al., “Effect of physical inactivity on major non-communicable diseases worldwide: an analysis of burden of disease and life expectancy” 380 LANCET 219 (2012); Schmid et al., “Association between physical activity and mortality among breast cancer and colorectal cancer survivors: a systematic review and meta-analysis” 25 ANN. ONCOL. 1293 (2014). For instance, short-term exercise training partially reverses the progression of metabolic diseases, whereas lifestyle interventions incorporating increased physical activity remain the primary preventive approach for metabolic diseases. In fact, regular exercise combined with dietary intervention is more successful than pharmacological intervention in the treatment of Type II Diabetes Mellitus (“T2DM”) and sarcopenia. Knowler et al., “Reduction in the incidence of type 2 diabetes with lifestyle intervention or metformin” 346 N. ENG. J. MED. 393 (2002); Borst, “Interventions for sarcopenia and muscle weakness in older people”, 33 AGE AGEING 548 (2004). While the benefits of adopting regular exercise are long known, molecular biologists have recently uncovered networks of signaling pathways and regulatory molecules that coordinate adaptive responses to exercise. Schmidt, supra at 1293; Mathieu-Costello et al., “Muscle structural capacity for oxygen flux from capillary to fiber mitochondria” 30 EXERC. SPORT SCI. REV. 80 (2002). Adaptation induced by exercise training results in the expansion of skeletal muscle vascularity and is recognized to be an important means of increasing the conductance of oxygen within exercising muscle. Schmid, supra at 1293; Mathieu-Costello, supra at 80. Exercise is known to directly impact the ECM in different tissues, Exercise has been reported to alter the collagen pattern in the heart of exercising rats. Kwak, “Aging, exercise, and extracellular matrix in the heart” 9 J. EXERC. REHABIL. 338 (2013).

Collagen degradation has been examined as a possible explanation for bone (Woitge et al., “Markers of bone and cartilage turnover” Exp Clin Endocrinol Diabetes 2017;125(7):454-69), tendon (Nogueira et al, “The effect of muscle actions on the level of connective tissue damage” Res Sport Med. 2011; 19(4):259-70), and muscle -related injuries (Mackey A L et al., “Skeletal muscle collagen content in humans after high-force eccentric contractions” J Appl Physiol. 2004; 97(1):197-203).

The amino acid hydroxyproline (HYP) is a major amino acid component of the protein collagen and helps stabilize collagen's tertiary structure. Hydroxyproline is commonly used as an indirect biomarker of collagen degradation and the integrity of connective tissue following high-intensity exercise. Nogueira, supra, Res Sport Med. 2011; 19(4):259-70; Minisola et al., “Clinical significance of free plasma hydroxyproline measurement in metabolic bone disease” Clin Chem Lab Med 1985; 23(9):515-20; Horswill et al., “Excretion of 3-methylhistidine arid hydroxyproline following acute weight-training exercise” Int J Sports Med 1988; 9(4):245-8; Suominen et al., “Effects of physical training on metabolism of connective tissues in young mice” Acta Physiol Scand 1980; 108(1):17-22; Tofas et al., “Plyometric exercise Increases serum indices of muscle damage and collagen breakdown” J Strength Cond Res 2008; 22(2):490-6.

There are a variety of collagen supplements available in the market, for oral ingestion as well as topical application, for instance to improve elasticity and firmness of aging skin and for improvement of joint health.

Chandan K. Sen (US 2016/0095881; “Promoting muscle building and repair and treating disorders related to collagen and pertinent proteins by using Shilajit”) describes a method of using Shilajit or its individual components, or a combination of two or more of these components, to induce the body of a mammal, Including that of a human, to synthesize new collagen and related extracellular matrix proteins, thus promoting the health of all tissues and organs containing collagen and related extracellular matrix proteins, including skin, connective tissue, muscle, cartilage, bone, and teeth and improve muscle building and regeneration, and/or treat collagen-related disorders.

Shilajit, also known as “Moomiyo,” is found in high altitudes, for instance of the Himalayan Mountains, and is considered as one of the “wonder medicines” of Ayurveda, the traditional Indian system of medicine dating back to 3500 B.C.E. Shilajit is regarded as one of the most important components in the Ayurvedic System of medicine and is also used as an adaptogen. Ghosal et al., “Shilajit I: chemical constituents” 65 J. PHARM. SCI. 772 (1976); Velmurugan et al., “Evaluation of safety profile of black shilajit after 91 days repeated administration in rats” 2 ASIAN PAC. J. TROP. BIOMED. 210 (2012). Shilajit is regarded as a “maharasa” (super-vitalizer) in Ayurveda. Shilajit is physiologically active organic matter, composed of rock humus, rock minerals, and organic substances that have been compressed by layers of rock mixed with marine organisms and microbial metabolites. Shilajit oozes out of the rocks as a black mass in the Himalayas at higher altitudes ranging from 1000 to 5000 meters, as the rocks become warm during summer. Velmurugan, supra at 210. Shilajit contains fulvic acids (“FA”) as its main components, along with dibenzo-α-pyrones (“DBPs”) and DBP chromoproteins, humic acid, and more than forty (40) minerals. DBPs are also known as Urolithins (e.g. Urolitin A, Urolithin B, etc.).

Shilajit finds extensive use in Ayurveda for diverse clinical conditions. For centuries, people living in the isolated villages in Himalaya and adjoining regions have used Shilajit alone, or in combination with other plant remedies, to prevent and combat problems with diabetes. Tiwari et al., “An interpretation of Ayurvedica findings on Shilajit” 8 J. RES. INDIGENOUS MED. 57 (1973) 8:57. Moreover, being an antioxidant, Shilajit will prevent pancreatic islet cell damage, which is induced by cytotoxic oxygen radicals. See, Bhattacharya, “Shilajit attenuates streptozotocin induced diabetes mellitus and decrease in pancreatic islet superoxide dismutase activity in rats” Phytother. Res. (1995) 9:41-4; Bhattacharya, “Effects of Shilajit on biogenic free radicals,” Phytother. Res. (1995) 9:56-9; and Ghosal et al., “Interaction of Shilajit with biogenic free radicals,” Indian J. Chem. (1995) 34B:596-602. It has been proposed that the derangement of glucose, fat, and protein metabolism during diabetes results in the development of hyperlipidemia. In one study, Shilajit produced significant beneficial effects in the lipid profile in rats. Trivedi et al., “Effect of Shilajit on blood glucose and lipid profile in alloxan-induced diabetic rats” Indian J. Pharmacal. (2004) 36(6):373-376). Oral supplementation with Shilajit significantly improved physiological energy status in albino mice In a model of forced swimming test. Bhattacharya, “Beneficial effect of processed Shilajit on swimming exercise induced impaired energy status of mice” PHARMACOLOGY ONLINE 817 (2009).

As discussed, Shilajit has been used to treat various ailments. It is also recommended as a performance enhancer. In addition to functioning as electrolytes and antioxidants, FAs are reported to elicit many important effects in the biological systems of plants and animals including humans, such as: (a) improvement of the bioavailability of minerals and nutrients; (b) detoxification of toxic substances including heavy metals; and (c) improvement of immune function.

Further, DBPs in Shilajit have been hypothesized to participate in the electron transport inside mitochondria, facilitating production of ATP (adenosine triphosphate), leading to increased energy. Bhattacharya et al., “Shilajit dibenzo-α-pyrones: Mitochondria targeted antioxidants” PHARMACOLOGYONLINE 690-698 (2009). Thus, Shilajit is found to increase energy, among other beneficial quantities.

Nutritional supplements that contain ingredients from traditional Ayurvedic medicine have garnered substantial interest related to muscle function and connection tissue health. Das et al., “The human skeletal muscle transcriptome in response to oral Shilajit supplementation” J Med Food 2016; 19(7):701-9; Wankhede et al., “Examining the effect of Withania somnifera supplementation on muscle strength and recovery: a randomized controlled trial.” J Int Soc Sports Nutr 2015; 12(1):43; Wilson et al, “Review on shilajit used in traditional Indian medicine” J Ethnopharmacol 2011; 136(1):1-9.

In addition to increasing adenosine triphosphate (ATP) availability via improved mitochondria function in mice (Bhattacharyya et al., “Beneficial effect of processed shilajit on swimming exercise induced impaired energy status of mice” Pharmacologyonline 2009; 1:817-25), recent studies have reported that supplementation with a purified, organic form of Shilajit (PrimaVie® Shilajit) increased free testosterone, total testosterone, and dehydroepiandrosterone by 19-31% in healthy men (Pandit et al., “Clinical evaluation of purified Shilajit on testosterone levels in healthy volunteers” Andrologia 2016; 48(5):570-5), which promotes increases in lean mass and muscular strength. Das et al. (2016), supra; Tapper et al., “Muscles of the trunk and pelvis are responsive to testosterone administration: data from testosterone dose—response study in young healthy men” Andrology 2018; 6(064-73; Bhasin et al., “The effects of supraphysiologic doses of testosterone on muscle size and strength in normal men” N Engl J Med 1996; 4; 335(1):1-7.

Recently, Das et al. (supra, J Med Food 2016; 19(7):701-9) reported that consuming 500 mg·d⁻¹ of PrimaVie® Shilajit for 8 weeks upregulated extracellular matrix (ECM)-related gene expression, which promotes collagen and connective tissue integrity.

### SUMMARY OF INVENTION

The present invention is directed to a method for reducing collagen degradation in a subject, and reducing muscle fatigue under fatiguing conditions, with the administration of Shilajit. Preferably, PrimaVie® Shilajit is used in the present methods.

The administration of Shilajit compositions according to the present invention significantly reduces muscle fatigue under fatiguing conditions, by reducing the decline in muscle strength due to muscle fatigue as measured by MVIC, and thus retaining muscle strength under fatiguing conditions. Also, the administration of Shilajit compositions according to the present invention significantly reduces serum hydroxyproline (HYP), an indicator of collagen degradation. Without being bound by theory, by reducing collagen degradation, the present invention increases collagen levels in tissues thereby improving muscle strength and elasticity, muscle building and repair, and improves the health of and/or treats diseases of skin, cartilage, connective tissues, muscle, vascular tissues, bones, and teeth.

In an embodiment, a method of the present invention is directed to reducing collagen degradation in a subject comprising the steps of (a) providing a composition comprising Shilajit, or its individual components, or a combination of at least (i) and (ii) below, the Shilajit or individual components or combination comprising (i) at least 10.3% by weight combined of free dibenzo-α pyrones and dibenzo-α-pyrones conjugated with chromoproteins and (ii) at least 50% by weight of fulvic acids with dibenzo-α-pyrone core nucleus; (b) administering the composition to the subject to deliver the Shilajit or its individual components or combination to the subject's bloodstream and bodily tissues in an amount effective to reduce collagen degradation in the tissues of the subject, and (c) reducing collagen degradation in the tissues of the subject. In an embodiment, in step (c), the Shilajit acts on said tissues to reduce collagen degradation.

In an embodiment, the present invention is directed to a method of improving skeletal muscle tissue and/or connective tissue in a subject comprising the steps of (a) providing a composition comprising Shilajit, or its individual components, or a combination of at least (i) and (ii) below, the Shilajit or individual components or combination comprising (i) at least 10.3% combined by weight of free dibenzo-α-pyrones and dibenzo-α-pyrones conjugated with chromoproteins, and (ii) at least 50% by weight of fulvic acids with dibenzo-α-pyrone core nucleus; (b) administering an effective amount of the Shilajit or individual components or combination to the subject to deliver the Shilajit or individual components or combination to the subject's bloodstream and skeletal muscle tissue and/or associated connective tissue; and (c) reducing collagen degradation in the subject's skeletal muscle tissue and/or associated connective tissue to improving the muscle tissue and/or connective tissue. This method may further include other steps, such as reducing muscle fatigue. In an embodiment, in step (c), the Shilajit acts on said tissues to reduce collagen degradation.

In an embodiment, the present invention is directed to a method of reducing muscle fatigue in a subject comprising the steps of (a) providing a composition comprising Shilajit, or its individual components, or a combination of at least (i) and (ii) below, the Shilajit or individual components or combination comprising (i) at least 10.3% combined by weight of free dibenzo-α-pyrones arid dibenzo-α-pyrones conjugated with chromoproteins, and (ii) at least 50% by weight of fulvic acids with dibenzo-α-pyrone core nucleus; (b) administering an effective amount of the Shilajit or individual components or combination to the subject to deliver the Shilajit or individual components or combination to the subject's bloodstream and skeletal muscle tissue and/or associated connective tissue; (c) reducing collagen degradation in the subject's skeletal muscle tissue and/or associated connective tissue to improve the muscle tissue; (d) introducing fatiguing conditions to the subject's skeletal muscle tissue; and (e) reducing muscle fatigue from the fatiguing conditions and retaining muscle strength as a result of the administration and delivery of the Shilajit or individual components or combination to the muscle tissue. In an embodiment, in step (c), the Shilajit acts on said tissues to reduce collagen degradation. in step (e), the Shilajit may act on the tissues as in step (c), or may optionally provide additional actions on the tissues.

Without being bound by theory, Shilajit such as Shilajit PrimaVie® improves mitochondrial function in the skeletal muscle of a subject, helping to improve for instance muscle strength and other muscle functions overall, and also to reduce muscle fatigue under fatiguing conditions.

An effective amount of Shilajit PrimaVie® allows the Shilajit to be delivered to the subject's bloodstream and tissues, i.e. to enter a subject's bloodstream and reach the subject's bodily tissues and act on those tissues, including for instance skeletal muscle tissue, connective tissue including cartilage, tendon, ligaments, vascular tissue, bones, and so forth. In an embodiment, an effective amount of Shilajit to reduce collagen degradation and/or muscle fatigue for a human subject is about 50 to about 1500 mg, 100-1000 mg, or 200-500 mg of PrimaVie® Shilajit. The same effective amount may be used in a subject having osteoporosis or arthritis, or to treat osteoporosis, arthritis, and/or symptoms thereof. In another embodiment, an effective amount of Shilajit is taken by the subject every day for at least 8 weeks.

In an embodiment, the method for reducing collagen degradation according to this invention reduces collagen degradation in connective tissue, and accordingly supports connective tissue health in the subject. For instance, the present methods reduce collagen degradation in tendons and/or ligaments, and accordingly improve tendon and ligament health in the subject. The reduction of collagen degradation according to the present invention supports the health and/or improves the skin, cartilage, muscle, vascular tissue, bone, and/or teeth of the subject. Methods of the present invention may be used in subjects having collagen-related disorders, including for instance to treat osteoporosis and/or arthritis, or symptoms thereof.

In an embodiment, a method of this invention further comprises the step of performing resistance training to increase the muscle mass and/or body weight of the subject, and so to increase muscular strength, size, and endurance. In an embodiment, a subject of the present invention is a recreational athlete.

### BRIEF DESCRIPTION OF DRAWINGS

FIG. 1 shows a timeline for the double-blind, placebo-controlled investigation described in the Example of the present application.

FIG. 2 shows a statistically significant (*) reduction in the decline of Maximal Voluntary Isometric Contraction (MVIC) strength after a fatiguing task and after 8 weeks' supplementation with PrimaVie® Shilajit, a standardized aqueous Shilajit extract, 500 mg/day (high dose). Low dose was 250 mg/day.

FIG. 3 shows a statistically significant (*) reduction in baseline serum hydroxyproline after 8 weeks supplementation with PrimaVie® Shilajit, a standardized aqueous Shilajit extract, 500 mg/day (high dose), Low dose was 250 mg/day.

### DETAILED DESCRIPTION

The utility of Shilajit for preventing or reducing collagen degradation and reducing muscle fatigue, and thus increasing muscular strength with resulting muscle building and regeneration, is completely novel and of tremendous value to mammals, including humans.

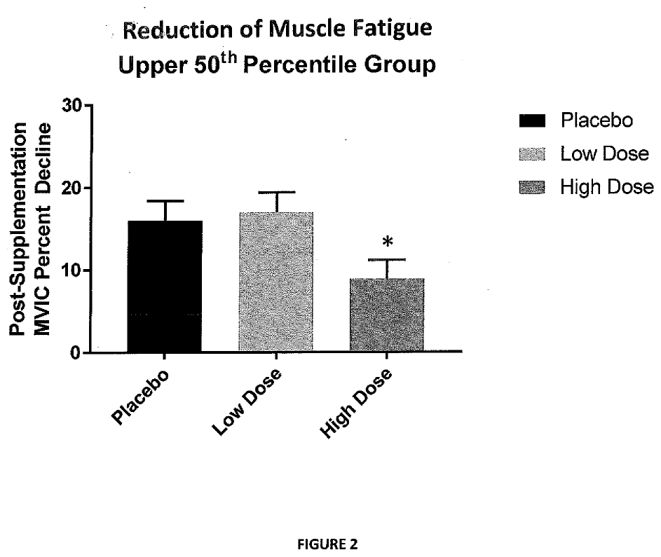

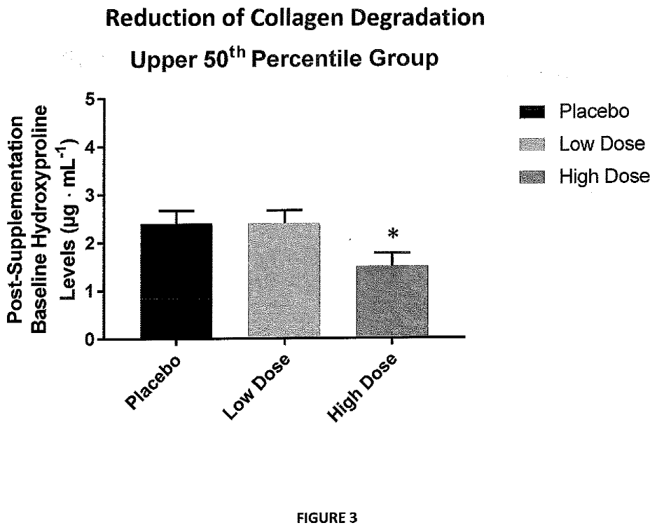

The administration of Shilajit compositions according to the present invention significantly reduces muscle fatigue under fatiguing conditions, as shown for instance by the 6% reduction in MVIC decline shown in FIG. 2. Also, as shown in FIG. 3, the administration of Shilajit compositions according to the present invention significantly reduces serum hydroxyproline (HYP), an indicator of collagen degradation, by 29%.

The below definitions and discussion are intended to guide understanding but are not intended to be limiting with regard to other disclosures in this application. References to percentage (%) in compositions of the present invention refers to the % by weight of a given component to the total weight of the composition being discussed, also signified by “w/w”, unless stated otherwise.

“Shilajit” is a rock exudate containing fulvic acids (“FAs”) as its main components, along with dibenzo-α-pyrones (“DSPs”) and DBP chromoproteins, humic acid, and more than forty (40) minerals. A composition of this invention comprises Shilajit, or its individual components, or a combination of at least (i) and (ii) below, the Shilajit or individual components or combination comprising and defined as (i) at least 10.3% by weight combined of free dibenzo-a pyrones and dibenzo-α-pyrones conjugated with chromoproteins and (ii) at least 50% by weight of fulvic acids with DBP core nucleus

In an embodiment, Shilajit according to this invention is a standardized aqueous extract of Shilajit (“standardized aqueous Shilajit extract”) containing at least 50% (w/w) fulvic acids with DRP core nucleus and at least 10.3% (w/w) of free DBP plus DBP conjugated with chromoproteins, and for instance more than 40 microminerals. The standardized aqueous Shilajit extract according to this invention is “PrimaVie® Shilajit”. PrimaVie® is a registered US trademark under which standardized aqueous Shilajit extract is sold, PrimaVie® Shilajit is described in U.S. Pat. Nos. 6,869,6:12 and 6,440,436, each of which is incorporated by reference herein for the purpose of describing PrimaVie® Shilajit.

In a further embodiment, said standardized aqueous Shilajit extract (“PrimaVie® Shilajit”) is a dry powder, having the appearance of a fine, brown to dark-brown, free-flowing powder; and further conforms to one or more of the following parameters: a water-soluble extractive value of at least 80% w/w; water in an amount of 0-6% (w/w); E₄/E₆ at 465/665 nm of 7.0-9.5; pH (2.0% aqueous dispersion) at least 5; lead at no more than 2 ppm, arsenic at no more than 3 ppm, and mercury at no more than 0.5 ppm; and a microbiological profile having not more than 5000 CFU/g aerobic bacteria (USP<2021>), not more than 1000 CFU/g yeast and mold (USP<2021>), with Escherichia coli (AOAC 991.14), Pseudomonas aeruginosa (USP<62>), Staphylococcus aureus (USP<2022>), and Candida albicans (custom) all absent in 1 g of said dry powder and Salmonella species (modified AOAC 998.09) absent in 10 g of said dry powder. In an embodiment, the standardized aqueous Shilajit extract of the present invention conforms to all of these parameters.

In a further embodiment, said standardized aqueous Shilajit extract is in dry powder form and further conforms to one or more of the following parameters: a water-soluble extractive value of about 93-96% (w/w), combined DBPs of about 14-17% (w/w), Fulvic acids with DBP core nucleus of about 59-62% (w/w), water in an amount of about 1-4% (w/w), E₄/E₆ at 465/665 nm at about 7-8, pH (2.0% aqueous dispersion) at about 6-8, lead in an amount less than 1 ppm, arsenic in an amount less than 0.6 ppm, mercury in an amount less than 0.01 ppm, less than about 500 CFU/g aerobic bacteria, less than 100 CFU/g yeast and mold, with E. coli, Salmonella, Pseudomonas aeruginosa, Staphylococcus aureus, and Candida albicans not measurably present. In an embodiment, the standardized aqueous Shilajit extract of the present invention conforms to all of these parameters.

In a further embodiment, said standardized aqueous Shilajit extract is in dry powder form and conforms to one or more of the following parameters: a water-soluble extractive value of 94.5% (w/w), combined DBPs of 15.67% (w/w), fulvic acids with DBP core nucleus of 60.81% (w/w), water in an amount of 2.75% (w/w), E₄/E₆ at 465/665 nm of 7.22, pH (2.0% aqueous dispersion) of 7.41, lead in an amount of 0.882 ppm, arsenic in an amount of 0.406 ppm, mercury in an amount less than 0.004 ppm, aerobic bacteria measured at 250 CFU/g or less, and yeast and mold at less than 70 CFU/g. E. coli, Salmonella, Pseudomonas aeruginosa, Staphylococcus aureus, and Candida albicans are not measurably present. In an embodiment, the standardized aqueous Shilajit extract of the present invention conforms to all of these parameters.

HPLC (High Pressure Liquid Chromatography) and HPTLC (High Pressure Thin Layer Chromatography) may be used to confirm the conformance of a standardized aqueous Shilajit extract of the present invention to e.g. fulvic acid and DBP parameters above, and other parameters as appropriate. In an embodiment, the standardized aqueous Shilajit extract in dry powder form is stable for 3 years or more. In an embodiment, the powdered extract is stored at 15° C. to 25° C., and in a container that avoids exposure of the powdered extract to light.

In an embodiment, standardized aqueous Shilajit extract (PrimaVie® Shilajit) is for nutraceutical use. In an embodiment, standardized aqueous Shilajit extract (PrimaVie® Shilajit) is for pharmaceutical use. In an embodiment, a composition of this invention is a standardized aqueous Shilajit extract as discussed above (PrimaVie® Shilajit). In another embodiment, a composition of this invention comprises the standardized aqueous Shilajit extract discussed above (PrimaVie® Shilajit).

In addition, a composition of the present invention may include excipients including for instance gelatin, microcrystalline cellulose, croscarmellose sodium, silicon dioxide, and/or magnesium stearate.

Shilajit or its equivalent(s) in embodiments of the present invention may be formulated into compositions including nutraceutical or pharmaceutical dosage forms comprising tablets, capsules, powders, liquids, chews, gummies, transdermals, injectables, etc, using standard excipients and formulation techniques in the industry.

A composition of the present invention as disclosed for instance in the below Examples may be made for instance according to the following steps: blending ingredients (except magnesium stearate, if present) in a 16 quart blender equipped with a sift-and-blend option and a 30 mesh stainless steel screen attachment for 15 minutes with screening at 1500 rpm. If any lumps remain, blending should continue for an additional 10 minutes at 1500 rpm to remove the lumps. Thereafter, magnesium stearate may be added to the blend, and the resulting powder blended for 5 minutes without using a sift-and-blend option. The blended composition may be stored in sealed containers, for instance avoiding light, for instance at 15° C.-25° C., for up to 3 years. In an embodiment, the composition is prepared under conditions of less than 50% relative humidity. In another embodiment, the composition is prepared under conditions of less than 40% relative humidity.

In an embodiment, a composition of the present invention such as the above blended composition may be encapsulated by filling capsules with an amount of the blended composition that will provide for instance 125 mg, 250 mg, 500 mg, or more of the purified Shilajit dry powder extract in the capsule. Other amounts of Shilajit (or its components or the combination with (i) and (ii) as defined above) may be included in the capsules or other delivery method. In an embodiment, a daily dose of Shilajit (or its components or combination as defined above) is provided as a discrete dose unit, or divided into 2 or more discrete dose units.

In an embodiment, a 125 mg Shilajit capsule may contain a blended composition comprising 125 mg standardized aqueous Shilajit extract (PrirnaVie® Shilajit), 100 mg microcrystalline cellulose (NF, Type 102), 10 mg croscarmellose sodium (NF), 2 mg silicon dioxide (NF, fumed, Cab-O-Sil®), 2 mg magnesium stearate (NF, vegetable derived). The total capsule fill weight of this composition is 239 mg. A type 1 capsule may be used, for instance a capsule weighing 74 mg, for a total capsule weight of 313 mg,

In an embodiment, a 250 mg Shilajit capsule may contain a blended composition comprising 250 mg standardized aqueous Shilajit extract (PrimaVie® Shilajit), 50 mg microcrystalline cellulose (NF, Type 102), 10 mg croscarmellose sodium (NF), 3 mg silicon dioxide (NF, fumed,)Cab-O-Sil®), 3 mg magnesium stea rate (NF, vegetable derived)). The total capsule fill weight of this composition is 316 mg. A type 1 capsule may be used, for instance a capsule weighing 74 mg, for a total capsule weight of 390 mg.

An “effective amount” or “amount effective . . . ” according to the present invention refers to an amount of Shilajit (or its individual components or a combination of its components as defined above), which when administered to a human or other subject, is effective in reducing and/or preventing collagen degradation in the subject's bodily tissues and/or reducing muscle fatigue in the subject's skeletal muscle tissue under fatiguing conditions. The present invention reduces the decline in strength typically accompanying muscle fatigue, thus retaining muscular strength under fatiguing conditions, and increases available collagen by reducing collagen degradation, thereby supporting tissue health, and strength and elasticity. An “effective amount” of Shilajit according to the present invention will allow the Shilajit (or its individual components or combination as defined throughout this application) to reach the subject's bloodstream and tissue including skeletal muscle tissue and reduce muscle fatigue in the skeletal muscle after the muscle has worked under fatiguing conditions, as shown in FIG. 2. Also, the effective amount will reduce collagen degradation in tissues of the subject's body, as evidenced by the decline in hydroxyproline (HYP) serum levels identified in FIG. 3.

In an embodiment of a method of the present invention, an effective amount of Shilajit (or its individual components or a combination of its components as defined above) in a human subject is about 50-1500 mg per day, or 100-1000 mg per day, or another amount within these ranges. For instance, in an embodiment of a method of the present invention, an effective amount of Shilajit in a human subject is 200-500 mg/day, In an embodiment, the effective amount is 250 mg/day or 500 mg/day. In an embodiment, about 400-600 mg/day is an effective amount. In an embodiment, an effective amount to be administered to a human subject is 50 mg-2000 mg, including 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, and any range including the above. In another embodiment, an effective amount is administered daily for at least 1 day, 1 week, 2 weeks, 3 weeks, 4 weeks, 5 weeks, 6 weeks, 7 weeks, 8 weeks, 9 weeks, 10 weeks, 11 weeks, 12 weeks, 4 months, 5 months, and so on, and administered chronically (e.g. 6 months or more) if desired.

In an embodiment, the effective amount to be administered is calculated per kilogram of body weight of the subject. For instance, a 500 mg daily dose in a 94 kg human adult male athlete is about 5.3 mg Shilajit per kilogram. In an embodiment, at least 0.6 mg Shilajit is administered to a human subject per kilogram. A 1500 mg daily dose in a 120 kg subject such as an adult male athlete is about 12.5 mg/kg. A 50 mg dose in a 70 kg subject is about 0.7 mg/kg subject. A 250 mg dose in an 80 kg subject is about 3 mg Shilajit/kg. A 600 mg daily dose In a 50 kg female athlete is administered in an amount of about 12 mg Shilajit/kg female athlete. In an embodiment, about 1 to about 15 mg Shilajit per day, or about 3 to about 12 mg per day, is administered to a subject. In an embodiment, about 5.5-6.5 mg Shilajit/kg human or other subject, or 6 mg/kg, is administered. In an embodiment, when calculating a dose of Shilajit according to the present invention, the dose should be equivalent to providing 50-2000, or 100-1000, mg Shilajit to a 70-94 kg subject. For a 5 kg dog, a dose of 6 mg Shilajit/kg of the subject may be about 30 mg daily. Similarly, for a 700 kg horse, an effective amount of Shilajit may be 700 kg horse×10 mg Shilajit/kg=about 7 grams Shilajit daily for the horse. In an embodiment, a dose of 1-10 mg/kg, or 3-5 mg/kg, or 5-6 mg/kg, or 6 mg -10 mg Shilajit/kg subject is administered.

“Administering” or “administration” of a composition of the present invention or the like refers to introducing the composition (including Shilajit or its individual components or a combination of components as defined above, and preferably Shilajit PrimaVie®) into the body of the human or other mammalian subject, so that the Shilajit is delivered to the subject's bloodstream and tissues, exposing the tissues to the Shilajit so that the Shilajit may change the tissues from their pre-administration state. The Shilajit interacts with the tissues to reduce collagen degradation, as evidenced by the decrease in hydroxyproline shown in FIG. 3; and to reduce the loss of strength in fatigued skeletal muscle, as evidenced in FIG. 2 and the Example, thus retaining strength under fatiguing conditions. Administration may be by the subject or by another. References to “supplementation” for instance in the Example below exemplify the administration of PrimaView® Shilajit according to this application. According to the present invention, administering an effective amount of Shilajit (or its individual components or combination thereof) to a subject reduces collagen degradation for instance in connective tissue, and reduces muscle fatigue/improves retention of muscle strength under fatiguing conditions in muscle tissue. In an embodiment, the muscle tissue is skeletal muscle tissue. Shilajit may be administered to the subject orally or by other forms of administration such as parenteral or transdermal administration, so long as an effective amount of the Shilajit is made available to the tissues of the human or other subject.

A “dietary supplement” according to the present invention refers to a composition of the present invention which is administered as an addition to a subject's diet, which is not a natural or conventional food, which effectively reduces collagen degradation and/or reduces muscle fatigue when administered to the human or other mammal over a period of time. In an embodiment, a dietary supplement containing an effective amount of Shilajit according to the present invention is administered orally. In an embodiment, the dietary supplement is administered daily; in an embodiment, the dietary supplement is administered daily for 8 weeks or more, or for another period of time according to the present invention. A dietary supplement may be formulated into various forms, as discussed throughout this application.

“Connective tissue” according to the present invention refers to tissue that contains collagen and connects, supports, or separates other tissues or organs, typically having relatively few cells embedded in an amorphous matrix, with collagen and often other fibers. Connective tissue includes for instance tendons, ligaments, and the endomysium of muscle tissue.

“Collagen degradation” according to the present invention refers to the breakdown of the protein collagen in the body of a subject. Serum levels of hydroxyproline (HYP) are used as an indicator of collagen degradation in the present invention. Reducing collagen degradation according to the present invention retains collagen in a subject's bodily tissues, improving for instance muscle tissue and its performance under fatiguing conditions, muscle building, and muscle repair and improving the health of collagen-containing tissues such as skin, cartilage, connective tissue(s), muscle, vascular tissue(s), bone(s), and teeth in the body of a subject, in particular a human subject, through the administration of Shilajit.

The reduction of collagen degradation and hence retention of collagen in bodily tissues by Shilajit such as PrimaVie® Shilajit treats collagen-related disorders including for instance osteoporosis and arthritis, in effective amounts such as discussed above. Also as discussed above, osteoporosis is experienced with age and is associated with reduced levels of collagen in the skin and bones. In an embodiment, a method of the present invention treats osteoporosis by providing and administering a composition such as PrimaVie® Shilajit in an effective amount to a subject having osteoporosis, so that the Shilajit bioactives enter the bloodstream and bodily tissues such as skin, bone, and associated connective tissues, and decrease collagen degradation in those tissues, as shown by the reduction of serum hydroxyproline (HYP) in subjects that have taken PrimaVie® Shilajit. By reducing collagen degradation in these tissues, the present methods increase the amount of collagen in the tissue, slowing and/or stopping the progress of the disease, and hence treating osteoporosis in the subject. The present methods may further treat osteoporosis for instance by retaining muscle strength, as discussed throughout this application.

As discussed above, arthritis is an inflammation of the joints, causing swelling and pain, and gradually degrades collagen in the joints, In an embodiment, a method of the present invention treats arthritis by providing and administering a composition such as PrimaVie® Shilajit in an effective amount to a subject having arthritis, so that the Shilajit bioactives enter the bloodstream and bodily tissues such as cartilage, bone, and associated tissues such as ligaments, tendons, and blood vessels, by reducing collagen degradation in those tissues, as shown by the reduction of serum hydroxyproline (HYP) in subjects that have taken PrimaVie® Shilajit. By reducing collagen degradation in these tissues, the present methods increase the amount of collagen in the tissue, slowing and/or stopping the progress of the disease, and hence treating arthritis in the subject. The present methods may further treat arthritis for instance by retaining muscle strength, as discussed throughout this application.

Collagen is a structural protein in humans as well as other mammals such as dogs and horses. In an embodiment, an effective amount of Shilajit for treating collagen-related disorders such as osteoporosis or arthritis is the same as an effective amount for reducing collagen degradation and/or reducing muscle fatigue and retaining muscle strength under fatiguing conditions according to the present invention—in humans, about 50 mg Shilajit to about 1500 mg Shilajit daily; in other mammals, about 1-22 mg/kg. In an embodiment, the effective amount of Shilajit for treating a non-human mammal such as a dog or horse is calculated as mg Shilajit per kg of subject.

A subject of the present invention that may be administered Shilajit or its individual components or a combination of said components, preferably PrimaVie® Shilajit, is a human or other mammal, such as a horse or dog. In an embodiment, the subject is a human. In an embodiment, the subject is an adult male human. In an embodiment, the subject is an adult female human. In an embodiment, the subject is about 70-95 kg, as in the below Example. In an embodiment, a subject according to the present invention is a human adult having strength as measured by MVIC of at least 350-360 N-m, for instance 355-560 N-m, or 400-550 N-m, as measured in the Example below, prior to Shilajit supplementation. In an embodiment, a subject according to the present invention is a human adult having an MVIC strength of at least 210-560 N-m, as measured in the Example below, prior to Shilajit supplementation. In an embodiment, a subject according to the present invention is a human adult having a baseline, pre-administration serum concentration of hydroxyproline of at least 0.65 ug per mL, for instance in the amount of 0.65-5 ug/ml. In an embodiment, a subject according to the present invention is a human adult having a baseline, pre-administration serum concentration of hydroxyproline of at least 1.85 ug per ml., preferably at least 2 ug per mL, and for instance in the range of 2-5 ug HYP per mL, Baseline serum hydroxyproline may be reduced in a subject of this invention, for instance by 29% as discussed above and shown in FIG. 2, and also for instance by 10-77%, along with other subranges within this range. A subject of the present invention may have osteoporosis and/or arthritis.

An “athlete” or “athletic subject” according to the present invention refers to a human subject that regularly exercises or plays sports, recreationally or professionally. In an embodiment, a subject according to the present invention is an athletic subject as described in the below Example.

“Physical activity” according to the present invention refers to activity such as exercise, sports, and/or work such as physical labor by a human.

“Muscle fatigue” or “fatigue” according to the present invention refers to a decrease in skeletal muscle strength during and/or after fatiguing conditions. Fatiguing conditions include repeated and/or extended physical activity using skeletal muscle such as exercise, sports, and/or work. In an embodiment, fatiguing conditions include the fatiguing protocol employed in the Example described herein: 2 bouts of 50 consecutive maximal, bilateral, concentric, isokinetic leg extension muscle actions. “Muscle strength” and the like according to the present invention refers to the maximum or near-maximum force a muscle can exert against external resistance, for instance in a single effort. Muscle strength is measured in an embodiment of the present invention as MVIC in the below Example. The loss of muscle strength due to muscle fatigue in a subject administered Shilajit in accordance with the present invention is reduced, for instance as shown in the below Example and FIG. 2, In an embodiment, after Shilajit administration according to this invention, a subject will retain at least 6% more muscle strength under fatiguing conditions than prior to Shilajit administration. For instance, if prior to Shilajit administration a subject's muscle strength declines 15% due to muscle fatigue from fatiguing conditions as measured by MVIC, then after Shilajit administration, the subject's muscle strength declines only 9% due to muscle fatigue. in another embodiment, after administration of Shilajit, a subject retains at least 6%, for instance 6-25%, or for instance 6%, 8%, 10%, 15%, 20%, 25%, or more muscle strength under fatiguing conditions after administration of Shilajit. Put another way, after administration of Shilajit according to this invention, muscle fatigue from fatiguing conditions, that is the loss of strength due to fatiguing conditions, is reduced by 6-25% or for instance 6%, 8%, 10%, 15%, 20%, 25% or more, as compared with pre-administration levels.

“Resistance training” according to the present invention refers to a type of exercise that improves muscle fitness including for instance muscle strength, and also for instance muscle power and/or endurance, by causing muscles to contract against external resistance. In an embodiment, the external resistance is provided by dumbbells, a weight-lifting machine, or an individual's own body weight.

“Health” according to the present invention generally refers to tissues of the subject that are functioning properly, that are regular and intact. For instance, as discussed above, healthy skin is firm, elastic, and hydrated, and undergoes reasonably prompt wound healing. Healthy bone is hard enough to perform its intended functions, and not weak or brittle. Cartilage, tendons, and ligaments are soft, fibrous, and elastic, not brittle or easily injured. In contrast, unhealthy, or even diseased, tissues include irregular or underperforming connective tissues, poor wound healing, brittle bones, deformities, and the like, for instance as discussed above regarding collagen-related disorders and degradation of collagen. Improvements in the health of tissues of the body, in their condition and status, may include improving healthy aspects of the tissue, such as improving the strength and/or elasticity of muscle, connective tissue, and/or skin, per the present methods.

The present invention may be further understood in connection with the following Example and embodiments. The following non-limiting Example and embodiments described throughout this application are provided to illustrate the invention.

### EXAMPLE

The effects of 8 weeks Shilajit supplementation (i.e. administration according to the present invention) at 250 mg·d⁻¹ (low dose) and 500 mg·d⁻¹ (high dose) versus placebo on maximal voluntary isometric contraction (MVIC) strength, fatigue-induced percent decline in strength, and serum HYP, were examined. This Example demonstrates at least that 8 weeks of PrimaVie® Shilajit supplementation at 500 mg·d⁻¹ promotes the retention of muscular strength following a fatiguing protocol and reduced baseline HYP. These findings are particularly associated with the stronger subjects and those with highest pre-supplementation levels of baseline HYP. Thus, 8 weeks of PrimaVie® Shilajit supplementation at 500 mg·d⁻¹ elicited favorable muscle and connective tissue adaptations.

### Methods

### Study Design

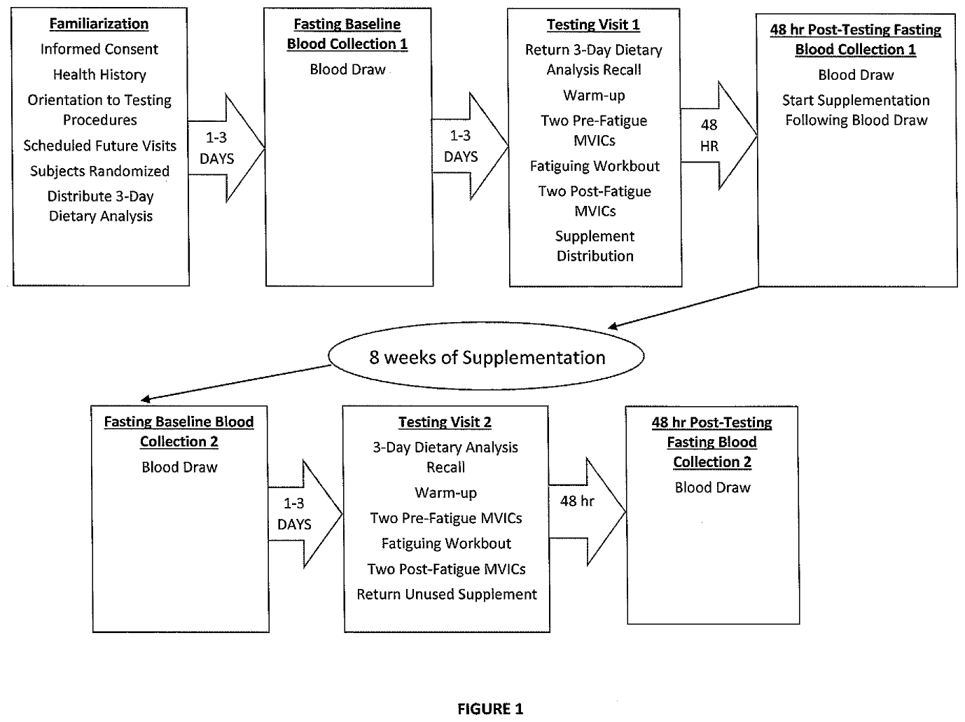

The present double-blind, placebo-controlled study followed the timeline in FIG. 1. The total sample (n=63) was randomized in three supplement groups: low dose Shilajit (n=21); high dose Shilajit (n=21); and placebo (n=21). The subjects visited the laboratory on seven occasions including a familiarization visit, two testing visits, and four fasting blood draws. The blood draws were to determine serum levels of HYP as an indicator of collagen degradation. The testing visits included pre- and post-fatigue measurements of bilateral, leg extension MVIC torque values. The fatiguing protocol involved two sets of 50 maximal, bilateral, concentric, isokinetic leg extension muscle actions at 180°·s⁻¹ separated by two minutes of rest. The two testing visits were separated by eight weeks of supplementation (FIG. 1).

### Subjects

Sixty-three recreationally trained (Pescatello L S. “Clinical exercise testing” ACSM's Guidelines for Exercise Testing and Prescription Philadelphia, Pa.: Wolters Kluwer/Lippincott Williams & Wilkins Health. 2014:114-41) men (X̅±SD: 21.2±2.4 yr; 179.8±6.3 cm; 83.1±12.7 kg) volunteered to participate in this study. The study was approved by The University Institutional Review Board for Human Subjects (IRB Approval #: 20171117645FB), and prior to testing all the subjects gave written Informed Consent and completed a health history questionnaire. The subjects in this study were involved in recreational sports such as rugby, basketball, softball, and flag-football and had no known cardiovascular, pulmonary, metabolic, or musculoskeletal diseases.

### Supplement and Supplementation

The subjects ingested a low dose (250 mg·d⁻¹ in one capsule) of PrimaVie® Shilajit (Natreon Inc., New Brunswick, N.J.), a high dose (500 mg·d⁻¹ in one capsule) of PrimaVie® Shilajit, or a placebo for eight weeks. The PrimaVie® Shilajit in the capsules was a purified and standardized aqueous extract of Shilajit in dry powder form as discussed above, having as bioactives 60.8% (w/w) fulvic acids with DBP core nucleus and 15.7% (w/w) of free DBP plus DBP conjugated with chromoproteins.

All capsules were Identical in size and appearance. Capsules containing PrimaVie® Shilajit, 250 mg or 500 mg, and placebo were ail supplied by Natreon, Inc. (New Brunswick, N.J.). All capsules included a blend of microcrystalline cellulose, croscarmellose sodium, silicon dioxide (fumed), and magnesium stearate, with Shilajit capsules further including 250 mg or 500 mg of the dry powder Shilajit extract described above.

250 mg Shilajit capsules contained, per unit dose capsule, a blended composition comprising 250 mg standardized aqueous Shilajit extract (PrimaVie® Shilajit), 50 mg microcrystalline cellulose (NF, Type 102), 10 mg croscarmellose sodium (NF), 3 mg silicon dioxide (NF, fumed, Cab-O-Sir), 3 mg magnesium stearate (NF, vegetable derived)). As the empty capsule (size 1) weighed 74 mg, the total weight of the 250 mg Shilajit capsules was 390 mg per capsule,

### Procedures

### Familiarization Visit

During the familiarization visit, the subjects performed submaximal (50-70% effort) and maximal, bilateral, isometric leg extension muscle actions as well as submaximal and maximal, bilateral, concentric, Isokinetic (180°·s⁻¹) leg extension muscle actions. All muscle actions were performed on a calibrated Cybex 6000 isokinetic dynamometer (Cybex International Inc. Medway, Mass.). Following the orientation, the subjects scheduled their first testing visit and were instructed to record their diet (MyFitnessPal, Inc.) during the three days before their scheduled testing visit 1. Also, the subjects scheduled their first fasting (12 hr overnight fast) baseline blood draw prior to their scheduled testing visit 1. The subjects were instructed to refrain from exercise for 48-hrs prior to testing visit 1,

### Testing Visit 1

All testing visits were scheduled before noon, and upon arrival/ each subject returned his 3-day dietary recall. The subjects performed a standard warm-up of 10 bilateral, isokinetic (180°·s⁻¹) leg extension muscle actions at a self-selected intensity of approximately 50-70% effort, Following 2 min of rest, the subjects performed 2, 6 s pretest MVICs at a knee joint angle of 120° (where 180° corresponds to full leg extension at the knee). Following the determination of MVIC, the subjects completed a fatiguing protocol that consisted of 2 bouts of 50 consecutive maximal, bilateral, concentric, isokinetic leg extension muscle actions separated by 2 min of rest. Each concentric muscle action was performed through a 90° range of motion (90° to 180° of leg extension). Thus, a total of 100 maximal, bilateral, concentric, isokinetic leg extension muscle actions were completed within a 5 min period. Following the fatiguing protocol, the subjects completed 2, 6 s posttest MVICs utilizing the same procedures as the pretest MVIC trial. Following the testing visit 1 exercise procedures, the subjects were scheduled to complete a fasted (12 hr overnight) 48-hr post-exercise blood draw. In addition, the subjects were instructed not to perform additional exercise between the testing visit 1 and the 48-hr post-exercise blood draw visit.

### 48 hr Post-Exercise Blood Draw Visit

Upon arrival, the subjects confirmed adherence to the overnight fast as well as not performing additional exercise since testing visit 1. Following confirmation, a fasted blood sample was collected, and the subjects started supplementation. Furthermore, the subjects were instructed to take the supplement once daily far 8 weeks (56 days) and to maintain their typical exercise and dietary habits.

8 weeks Shilalit Supplementation

Throughout the 8 weeks of Shilajit supplementation, the subjects were contacted biweekly to promote adherence to supplementation as well as to ensure that no subjects had experienced any adverse effects possibly related to the supplement. During the last week of supplementation, the subjects scheduled the post-supplementation baseline blood draw visit as well as testing visit 2. The subjects were reminded to fast overnight prior to the post-supplementation baseline blood collection visit, to avoid performing any exercise 48-hr prior to testing visit 2, and to record their diet for 3 days before testing visit 2.

### Testing Visit 2

Following the 8 weeks of Shilajit supplementation and the post-supplementation baseline blood draw visit, the subjects returned to the laboratory and provided their 3-day dietary analysis, The subjects then repeated the same testing procedures as testing visit 1. Following the completion of all testing procedures, the subjects were scheduled for their 48 hr past-exercise blood collection. Again, the subjects were instructed not to exercise and to complete an overnight 12 hr fast prior to the 48 hr post-exercise blood draw. Furthermore, the subjects returned any remaining supplement, so that adherence could be documented.

### Determination of Strength, Percent Decline, and Collagen Degradation

During testing visits 1 and 2, concentric peak torque, MVIC, percent decline in MVIC, and percent decline in peak torque were determined. For concentric peak torque, the mean of the first five repetitions of the first bout of 50 maximal, bilateral, concentric isokinetic leg extension muscle actions was defined as pretest concentric peak torque, and the mean of the last five repetitions of the second bout of 50 maximal, bilateral, concentric isokinetic leg extension muscle actions was defined as posttest concentric peak torque. In addition, pretest MVIC and posttest MVIC were defined as the greatest torque produced during the 2 MVIC muscle actions for each test. Furthermore, percent decline in MVIC and percent decline in peak torque were determined with the following equations:

$$\begin{matrix} Percent Decline = \frac{Pretest MVIC - Posttest MVIC}{Pretest MVIC} * 100 & EQN ( 1 ) \\ Percent Decline = \frac{Pretest Peak Torque - Posttest Peak Torque}{Pretest Peak Torque} * 100. & EQN ( 2 )\end{matrix}$$

Each subject provided 8 mL of whole blood from the antecubital vein at pre-supplementation baseline and 48 hrs post-exercise as well as at post-supplementation baseline and 48 hrs post-exercise. Thus, there was a total of 252 (4 samples per subject) blood samples. Following the blood draws, the samples were each centrifuged and stored at −80° C. Subsequently, 100 μL of each individual sample was aliquoted into 2 mL screw cap tubes and prepared for the tandem technique of high-performance liquid chromatography mass spectrometry, Furthermore, the column used was ACCQ-TAG Ultra C18 1.7 μm, 2.1×100 mm, with mobile phase A, 100% Eluent A, mobile phase B, 10:90 Eluent B: Milli-Q water, mobile phase C, Milli-Q water, and mobile phase 0, 100% Eluent B. The flow rate was at 0.7 mL/min, the column oven was at 45° C., and the runtime was 13 min per sample. Standard curves were used to calculate the concentration of HYP in the samples from the peak area detected, using the following formula:

Serum HYP Concentration (μg·mL⁻¹)= EQN (3)
(peak area HYP)/(peak area internal standard)•(Volume of internal standard•concentration of internal standard spike)/(slope from the standard curve•(molecular weight HYP)/1.000/volume of starting material)

### Statistical Analyses

Test-retest reliability for MVIC, concentric peak torque, and baseline HYP were examined for the pre-test, pre-supplementation versus pre-test, post-supplementation measurements. Repeated measures ANOVAs were used to evaluate systematic error, and the 2,k model (Weir, “Quantifying test-retest reliability using the intraclass correlation coefficient and the SEM” J Strength Cond Res 2005; 19(1):231-40) was used to determine the intraclass correlation coefficient (ICC) and standard error of the measurements (SEM).

Each dependent variable (MVIC, concentric peak torque, and HYP levels) was statistically analyzed using a 3 (Group: Low Dose, High Dose, Placebo)×2 (Visit: Testing Visit 1, Testing Visit 2)×2 (Fatigue: Pretest, Posttest) mixed factorial ANOVA to examine mean differences for absolute values. In addition, ANCOVA was used to examine post-supplementation differences between groups for percent decline in MVIC, percent decline in peak torque, percent change in baseline to 48 hrs post-exercise HYP levels, and percent change in baseline HYP levels covarying for pre-supplementation values. Huck S W et al., “Using a repeated measures ANOVA to analyze the data from a pretest-posttest design: A potentially confusing task” Psychol Bull 1975; 82(4):511; Vincent et al., “Statistics in Kinesiology. Human Kinetics” Champaign (IL): 2012. Furthermore, paired t-tests were used to examine pre-supplementation versus post-supplementation for total caloric intake, carbohydrates, proteins, and fats.

Additional analyses were performed separately for sub-samples of each group that were dichotomized at the 50^{th} percentile (median) for pre-supplementation MVIC, concentric peak torque, and baseline HYP levels.

Pre-supplementation measurements of MVIC ranged from about 200-560 N-m. The highest and strongest pre-supplementation MVIC measurements (upper 50^{th} percentile) ranged from about 360 N-m to about 560 N-m. In the lower 50^{th} percentile, MVIC strength ranged from about 215 N-m to about 350 N-m. See Tables 1A, 1B, and 1C for further data. The above ranges are taken from data from these tables. Similar or other ranges may be derived from the Tables as needed.

Pre-supplementation peak torque (Set 1) ranged from about 148-331. Upper and lower 50^{th} percentile groupings of subjects were the same as the groupings for MVIC measurements. See Tables 2A, 2B, and 2C for further data. The above range is taken from data from these tables. Similar or other ranges may be derived from the Tables as needed.

Pre-supplementation baseline serum HYP ranged from about 0.65 to about 5 ug/ml. Pre-supplementation baseline serum HYP levels in the upper 50th percentile ranged from 1.86-4.80 ug per mL, Pre-supplementation baseline serum HYP levels in the lower 50^{th} percentile ranged from about 0.7 to about 1.9 ug per mL. See Tables 3A, 3B, and 3C for further data. The above ranges are taken from data from these tables. Similar or other ranges may be derived from the Tables as needed.

For each dependent variable, a 3 (Group: Low Dose, High Dose, Placebo)×2 (Visit: Testing Visit 1, Testing Visit 2)×2 (Fatigue: Pretest, Posttest) mixed factorial ANOVA was used to examine mean differences in absolute values separately for the upper 50th percentile sub-sample (n=10 per group for each dependent variable) and the lower 50^{th} percentile sub-sample (n=11 per group for each dependent variable). Also, for each sub-sample, ANCOVAs were used to examine post-supplementation differences between groups for percent decline in MVIC, percent decline in peak torque, percent change in baseline to 48 hrs post-exercise HYP levels, and baseline HYP levels covaried for pre-supplementation values.

Therefore, a total of nine mixed factorial ANOVAs and twelve ANCOVAs were used to examine mean differences in MVIC, concentric peak torque, percent decline in MVIC, percent decline in concentric peak torque, and HYP levels. Furthermore, significant interactions were decomposed with follow-up repeated measures ANOVAs and independent or paired samples t-tests, Greenhouse-Geisser corrections were applied when sphericity was not met according to Maulchy's Test of Sphericity, and effect sizes were calculated for each comparison. Specifically, partial eta squared (ηₚ²) for each ANOVA and Cohen's d for each Student's t-test were calculated. All data is presented mean ±Standard Error of the Mean. In addition, all statistical analyses were performed using IBM SPSS v. 25 (Armonk, N.Y.) and an alpha of p<0,05 was considered statistically significant for all comparisons,

### Results

### Test-Retest Reliability

Test-retest reliability was quantified using the 2,k model of Weir (supra, J Strength Cond Res 2005; 19(1):231-40) for mean differences (systematic error), ICCs, and SEM measured pre-supplementation versus post supplementation (8 weeks apart) from the placebo group (n=21). There was no significant mean difference for test versus retest for MVIC (X̅±SD=366.3±81.8 Nm versus 346.0±63.6 Nm; p=0.108) or concentric peak torque (236.9±56.3 Nm versus 246.1±42.9 Nm; p=0.415). The ICC and SEM values for MVIC and concentric peak torque were 0.75 and 52.1 Nm and 0.66 and 41.5 Nm, respectively. Test-retest reliability for HYP was determined from the baseline HYP values measured pre-supplementation versus post-supplementation (8 weeks apart) for the placebo group (n=21). There was no significant mean difference for test versus retest for baseline HYP (X̅±SD=2.1±0.90 μg·mL⁻¹ versus 2.1±0.85 μg·mL⁻¹; p=0.622) the ICC=0.58, and the SEM=0.81 μg·mL⁻¹.

### Adverse Events, Dietary Analyses, and Subject Adherence

There were no reported adverse events during the course of this study. In addition, there were no significant (p>0.05) differences for the dietary analyses of total caloric, carbohydrate, protein, or fat intake for pre-supplementation versus post-supplementation or between groups. Based on the number of capsules taken over the 8 week supplementation period, subject adherence was 95% (53 out of 56 capsules), 95% (53 out of 56 capsules), and 96% (54 out of 56 capsules) for the low dose, high dose, and placebo groups, respectively.

### Absolute MVC Values and Percent Decline in MVIC

Total Group (n=63; 21 Per Group)

The 3 (Group: Low, High, and Placebo)×2 (Visit: Testing Visit 1 and Testing Visit 2)×2 (Fatigue: Pretest and Posttest) mixed factorial ANOVA indicated that there was no significant (p=0.267, ηₚ²=0.043) 3-way or 2-way interactions (p=0.215, ηₚ²=0.026; p=0.063, ηₚ²=0.088; p=0.955, ηₚ²=0.002). Furthermore, there was no significant (p=0.935, ηₚ²=0.002) main effect for Group. There was, however, a significant (p=0,005, ηₚ²=0.122) main effect for Fatigue (collapsed across Group and Visit), and the pairwise comparison indicated that Pretest MVIC (341.8±8.3 Nm) was significantly greater than Posttest MVIC (323.8±8.3 Nm).

The 1-way ANCOVA indicated that there was no significant (p=0.172, ηₚ²=0.031) difference for pre-supplementation percent decline in MVIC, but that there was a significant (p=0.047; ηₚ²=0.099) difference between groups for the adjusted (covaried for pre-supplementation values) mean post-supplement percent decline in MVIC. The pairwise comparisons indicated that the adjusted mean post-supplementation percent decline in MVIC was significantly (p=0.021) less for the high dose (9.1±2.0%) than the low dose (15.7±2.0%).

Upper 50^{th} Percentile (n=30; 10 Per Group)

The 3 (Group: Low, High, and Placebo)×2 (Visit; Testing Visit 1 and Testing Visit 2)×2 (Fatigue: Pretest and Posttest) mixed factorial ANOVA indicated that there was no significant (p=0.169, ηₚ²=0.123) 3-way or 2-way interactions containing Group (p=0.057, ηₚ²=0.191; p=0.070, ηₚ²=0.179). There was, however, a significant (p<0.001, ηₚ²=0.434) main effect for Fatigue (collapsed across Group and Visit), and the pairwise comparison indicated that Pretest MVIC (394.8±9.0 Nm) was significantly greater than Posttest MVIC (357.6±10.5 Nm). In addition, there was no significant (p=0.168, ηₚ²=0.168) main effect for Group.

The 1-way ANCOVA indicated that there was a significant (p=0.029, ηₚ²=0.170) difference between groups for pre-supplementation percent decline in MVIC and a significant (p=0.042; ηₚ²=0.217) difference between groups for the adjusted (covaried for pre-supplementation values) mean post-supplementation percent decline in MVIC. The pairwise comparisons indicated that the post-supplementation adjusted mean percent decline in MVIC was significantly (p=0.022 and 0.044) less for the high dose group (8.9±2.3%) than the low dose (17.0±2.4%) and placebo (16.0±2.4%) groups.

See FIG. 2; the asterisk (*) denotes the significant difference between the high dose group and the low dose and placebo groups. There was no significant (p=0.774) difference, however, between the low dose and placebo groups, FIG. 2 shows ANCOVA results for the adjusted (covaried for pre-supplementation values) mean scores of post-supplementation MVIC percent decline. Note: Bars are X̅±Standard Error of the Mean. MVIC=Maximal voluntary isometric contraction. *High dose (500 mg·d⁻¹ of PrimaVie® Shilajit)<low dose (250 mg·d⁻¹ of PrimaVie® Shilajit; p=0.022) and placebo (p=0.044).

Lower 50^{th} Percentile (n=33; 11 Per Group)

The 3 (Group: Low, High, and Placebo)×2 (Visit: Testing Visit 1 and Testing Visit 2)×2 (Fatigue: Pretest and Posttest) mixed factorial ANOVA indicated that there was no significant (p=0.825, η_{hu 2}=0.013) 3-way interaction or any significant 2-way interaction (p=0.992, ηₚ²<0.001; p=0.494, ηₚ²=0.046; p=0.154, ηₚ²=0.117). In addition, there was no significant (p=0.267, ηₚ²=0.084) main effect for Group.

The 1-way ANCOVA indicated that there was not a significant (p=0.853, ηₚ²=0.001) difference between groups for pre-supplementation percent decline in MVIC and no significant (p=0.445; ηₚ²=0.054) difference between groups for the adjusted (covaried for pre-supplementation values) mean post-supplementation percent decline in MVIC.

Tables 1A, 1B, and 1C show MVIC data for study participants in the Low Dose Group (250 mg Shilajit), High Dose Group (500 mg Shilajit), and Placebo Group (No Shilajit). MVIC measurements are in N-m. Study participants in the Upper 50^{th} percentile for their group are marked as Upper, and study participants in the Lower 50^{th} percentile for their group are marked as Lower. Individuals 1-21 numbered within the groups are numbered independently of study identifiers.

TABLE 1A

| | PreSuppl. | PreSuppl. | PostSuppl. | PostSuppl. | | |
|---|---|---|---|---|---|---|
| Low Dose | PreMVIC | PostMVIC | PreMVIC | PostMVIC | PreSuppl. | PostSuppl. |
| Group | (N-m) | (N-m) | (N-m) | (N-m) | % Decline | % Decline |
| UPPER 1 | 458 | 317 | 412 | 342 | 30.8 | 17.0 |
| UPPER 2 | 516 | 439 | 502 | 439 | 14.9 | 12.5 |
| UPPER 3 | 438 | 365 | 492 | 442 | 16.7 | 10.2 |
| UPPER 4 | 363 | 252 | 201 | 193 | 30.6 | 4.0 |
| UPPER 5 | 485 | 442 | 489 | 431 | 8.9 | 11.9 |
| UPPER 6 | 487 | 430 | 381 | 370 | 11.7 | 2.9 |
| UPPER 7 | 400 | 253 | 418 | 300 | 36.8 | 28.2 |
| UPPER 8 | 364 | 276 | 349 | 275 | 24.2 | 21.2 |
| UPPER 9 | 453 | 383 | 415 | 316 | 15.5 | 23.9 |
| UPPER 10 | 463 | 402 | 384 | 318 | 13.2 | 17.2 |
| UPPER 11 | 409 | 305 | 477 | 300 | 25.4 | 37.1 |
| LOWER 12 | 329 | 304 | 285 | 284 | 7.6 | 0.4 |
| LOWER 13 | 288 | 283 | 330 | 316 | 1.7 | 4.2 |
| LOWER 14 | 340 | 305 | 489 | 268 | 10.3 | 45.2 |
| LOWER 15 | 293 | 260 | 325 | 219 | 11.3 | 32.6 |
| LOWER 16 | 289 | 225 | 312 | 244 | 22.1 | 21.8 |
| LOWER 17 | 271 | 259 | 226 | 220 | 4.4 | 2.7 |
| LOWER 18 | 345 | 263 | 239 | 220 | 23.8 | 7.9 |
| LOWER 19 | 288 | 245 | 277 | 228 | 14.9 | 17.7 |
| LOWER 20 | 287 | 237 | 264 | 240 | 17.4 | 9.1 |
| LOWER 21 | 333 | 319 | 306 | 280 | 4.2 | 8.5 |

TABLE 1B

| | PreSuppl. | PreSuppl. | PostSuppl. | PostSuppl. | | |
|---|---|---|---|---|---|---|
| High Dose | PreMVIC | PostMVIC | PreMVIC | PostMVIC | PreSuppl. | PostSuppl. |
| Group | (N-m) | (N-m) | (N-m) | (N-m) | % Decline | % Decline |
| UPPER 1 | 391 | 357 | 320 | 290 | 8.7 | 9.4 |
| UPPER 2 | 356 | 306 | 392 | 305 | 14.0 | 22.2 |
| UPPER 3 | 465 | 404 | 472 | 465 | 13.1 | 1.5 |
| UPPER 4 | 410 | 375 | 373 | 358 | 8.5 | 4.0 |
| UPPER 5 | 360 | 313 | 313 | 271 | 13.1 | 13.4 |
| UPPER 6 | 418 | 287 | 320 | 313 | 31.3 | 2.2 |
| UPPER 7 | 405 | 320 | 333 | 258 | 21.0 | 22.5 |
| UPPER 8 | 505 | 435 | 411 | 409 | 13.9 | 0.5 |
| UPPER 9 | 375 | 340 | 266 | 249 | 9.3 | 6.4 |
| UPPER 10 | 381 | 353 | 389 | 343 | 7.3 | 11.8 |
| UPPER 11 | 388 | 235 | 319 | 261 | 39.4 | 18.2 |
| LOWER 12 | 331 | 309 | 314 | 306 | 6.6 | 2.5 |
| LOWER 13 | 306 | 242 | 296 | 268 | 20.9 | 9.5 |
| LOWER 14 | 299 | 213 | 293 | 264 | 28.8 | 9.9 |
| LOWER 15 | 325 | 277 | 249 | 224 | 14.8 | 10.0 |
| LOWER 16 | 351 | 329 | 326 | 313 | 6.3 | 4.0 |
| LOWER 17 | 310 | 299 | 388 | 319 | 3.5 | 17.8 |
| LOWER 18 | 315 | 290 | 345 | 336 | 7.9 | 2.6 |
| LOWER 19 | 333 | 319 | 311 | 281 | 4.2 | 9.6 |
| LOWER 20 | 337 | 297 | 276 | 245 | 11.9 | 11.2 |
| LOWER 21 | 326 | 289 | 335 | 334 | 11.3 | 0.3 |

TABLE 1C

| | PreSuppl. | PreSuppl. | PostSuppl. | PostSuppl. | | |
|---|---|---|---|---|---|---|
| Placebo | PreMVIC | PostMVIC | PreMVIC | PostMVIC | PreSuppl. | PostSuppl. |
| Group | (N-m) | (N-m) | (N-m) | (N-m) | % Decline | % Decline |
| UPPER 1 | 367 | 357 | 293 | 238 | 2.7 | 18.8 |
| UPPER 2 | 362 | 331 | 349 | 329 | 8.6 | 5.7 |
| UPPER 3 | 438 | 405 | 366 | 349 | 7.5 | 4.6 |
| UPPER 4 | 395 | 370 | 402 | 347 | 6.3 | 13.7 |
| UPPER 5 | 494 | 367 | 368 | 306 | 25.7 | 16.8 |
| UPPER 6 | 358 | 300 | 340 | 330 | 16.2 | 2.9 |
| UPPER 7 | 424 | 368 | 440 | 388 | 13.2 | 11.8 |
| UPPER 8 | 394 | 393 | 310 | 247 | 0.3 | 20.3 |
| UPPER 9 | 479 | 326 | 390 | 301 | 31.9 | 22.8 |
| UPPER 10 | 552 | 461 | 447 | 391 | 16.5 | 12.5 |
| UPPER 11 | 412 | 366 | 339 | 270 | 11.2 | 20.4 |
| LOWER 12 | 314 | 288 | 360 | 331 | 8.3 | 8.1 |
| LOWER 13 | 329 | 299 | 377 | 305 | 9.1 | 19.1 |
| LOWER 14 | 337 | 257 | 428 | 369 | 23.7 | 13.8 |
| LOWER 15 | 266 | 217 | 241 | 216 | 18.4 | 10.4 |
| LOWER 16 | 219 | 205 | 254 | 177 | 6.4 | 30.3 |
| LOWER 17 | 255 | 249 | 320 | 292 | 2.4 | 8.8 |
| LOWER 18 | 332 | 280 | 350 | 284 | 15.7 | 18.9 |
| LOWER 19 | 289 | 283 | 253 | 229 | 2.1 | 9.5 |
| LOWER 20 | 346 | 314 | 412 | 326 | 9.2 | 20.9 |
| LOWER 21 | 330 | 203 | 243 | 221 | 38.5 | 9.1 |

### Absolute Concentric Peak Torque and Percent Decline in Concentric Peak Torque

Total Group (n=63; 21 Per Group)

The 3 (Group: Low, High, and Placebo)×2 (Visit; Testing Visit 1 and Testing Visit 2)×2 (Fatigue: Pretest and Posttest) mixed factorial ANOVA indicated that there was no significant (p=0.185, ηₚ²=0.055) 3-way or 2-way interactions containing Group (p=0.582, ηₚ²=0.018; p=0.524, ηₚ²=0.021). In addition, there was no significant (p=0.572, ηₚ²=0.018) main effect for Group.

The 1-way ANCOVA indicated that there was a significant (p<0.001, ηₚ²=0.282) difference between groups for pre-supplementation percent decline in concentric peak torque, but there was no significant (p=0.274; ηₚ²=0.043) difference between groups for the adjusted (covaried for pre-supplementation values) mean post-supplementation percent decline in concentric peak torque.

Upper 50^{th} Percentile (n=30; 10 Per Group)

The 3 (Group: Low, High, and Placebo)×2 (Visit: Testing Visit 1 and Testing Visit 2)×2 (Fatigue: Pretest and Posttest) mixed factorial ANOVA indicated that there was a significant (p=0.004, ηₚ²=0.339) 3-way Interaction. Subsequently, three (1 for each group) follow-up 2 (Visit: Testing Visit 1 and Testing Visit 2)×2 (Fatigue: Pretest and Posttest) repeated measures ANOVAs were used. For the low dose group, the follow-up analyses indicated that there was a significant (p=0.049, ηₚ²=0.365) 2-way interaction, therefore, a paired t-test was used to examine pre-fatigue mean concentric peak torque at both pre- and post-supplementation. The t-test indicated that there was no significant (267.4±7.6 vs. 274.2±9.0 Nm; p=0.051, d=0.26) difference between pre-fatigue, pre-supplementation, peak, concentric torque and pre-fatigue, post-supplementation, peak, concentric torque. For the high dose group, the follow-analyses indicated that there was no significant (p<0.05) 2-way interaction or main effect related to fatigue. For the placebo group, the follow-up analyses indicated that there was a significant (p=0.022, ηₚ²=0.458) 2-way interaction, therefore, a paired t-test was used to examine pre-fatigue, concentric, peak torque at both pre- and post-supplementation. The t-test indicated that pre-fatigue, pre-supplementation, concentric, peak torque was significantly (283.3±11.5 vs. 254.5±13.7 Nm; p=0.008, d=0.72) greater than pre-fatigue, post-supplementation, peak, concentric torque.

The 1-way ANCOVA indicated that there was a significant (p<0.001, ηₚ²=0.622) difference between groups for pre-supplementation percent decline in concentric peak torque, but there was no significant (p=0.286; ηₚ²=0.092) difference between groups for the adjusted (covaried for pre-supplementation values) mean post-supplementation percent decline in concentric peak torque.

Lower 50^{th} Percentile (n=33; 11 Per Group)

The 3 (Group: Low, High, and Placebo)×2 (Visit: Testing Visit 1 and Testing Visit 2)×2 (Fatigue: Pretest and Posttest) mixed factorial ANOVA indicated that there was no significant (p=0.349, ηₚ²=0.068) 3-way or 2-way interaction containing Group. In addition, there was no main effect (p=0.126; ηₚ²=0.129) for Group.

The 1-way ANCOVA indicated that there was a significant (p=0.005, ηₚ²=0.240) difference between groups for pre-supplementation percent decline in concentric peak torque, but there was no significant (p=0.841; ηₚ²=0.0012) difference between groups for the adjusted (covaried for pre-supplementation values) mean post-supplementation percent decline in concentric peak torque.

TABLE 2A

| | PreSup | PreSup | | PreSup | PreSup | | | PstSup | PstSup | | PstSup | PstSup ! | | |
|---|---|---|---|---|---|---|---|---|---|---|---|---|---|---|
| | Peak | Last | PreSup | Peak | Last | PreSup | PreSup | Peak | Last | PstSup | Peak | Last | PstSup | PstSup |
| | Torque | Torque | Set1 | Torque | Torque | Set2 | Total | Torque | Torque | Set1 | Torque | Torque | Set2 | Total |
| Low Dose | Set1 | Set1 | % | Set2 | Set2 | % | % | Set1 | Set1 | % | Set2 | Set2 | % | % |
| (250 mg/ml) | (N-m) | (N-m) | Decline | (N-m) | (N-m) | Decline | Decline | (N-m) | (N-m) | Decline | (N-m) | (N-m) | Deciine | DecLine |
| UPPER1 | 233.7 | 145.3 | 37.8 | 167.0 | 55.3 | 66.9 | 76.3 | 311.7 | 130.3 | 58.2 | 243.3 | 111.0 | 54.4 | 64.4 |
| UPPER2 | 270.7 | 177.7 | 34.4 | 230.3 | 171.0 | 25.8 | 36.8 | 261.0 | 161.3 | 38.2 | 263.0 | 97.0 | 63.1 | 62.8 |
| UPPER3 | 230.0 | 182.7 | 20.6 | 244.0 | 157.7 | 35.4 | 31.4 | 345.0 | 158.0 | 54.2 | 274.7 | 127.0 | 53.8 | 63.2 |
| UPPER4 | 188.0 | 139.3 | 25.9 | 173.0 | 113.3 | 34.5 | 39.7 | 154.0 | 115.3 | 25.1 | 154.0 | 108.0 | 29.9 | 29.9 |
| UPPER5 | 263.7 | 197.3 | 25.2 | 297.0 | 163.0 | 45.1 | 38.2 | 277.7 | 188.0 | 32.3 | 276.7 | 206.0 | 25.5 | 25.8 |
| UPPER6 | 305.7 | 239.0 | 21.8 | 350.7 | 250.3 | 28.6 | 18.1 | 318.0 | 237.7 | 25.3 | 348.3 | 233.3 | 33.0 | 26.6 |
| UPPER7 | 207.3 | 161.3 | 22.2 | 154.0 | 95.7 | 37.9 | 53.9 | 224.7 | 135.7 | 39.6 | 166.7 | 150.7 | 9.6 | 32.9 |
| UPPER8 | 252.7 | 180.3 | 28.6 | 228.3 | 127.3 | 44.2 | 49.6 | 249.0 | 157.7 | 36.7 | 218.7 | 131.3 | 39.9 | 47.3 |
| UPPER9 | 247.3 | 161.7 | 34.6 | 270.0 | 122.3 | 54.7 | 50.5 | 261.3 | 128.3 | 50.9 | 223.0 | 158.3 | 29.0 | 39.4 |
| UPPER10 | 255.3 | 172.7 | 32.4 | 189.3 | 127.0 | 32.9 | 50.3 | 260.7 | 171.7 | 34.1 | 255.0 | 113.7 | 55.4 | 56.4 |
| UPPER11 | 314.3 | 175.7 | 44.1 | 297.0 | 113.0 | 62.0 | 64.1 | 333.3 | 137.0 | 58.9 | 267.7 | 98.7 | 63.1 | 70.4 |
| LOWER12 | 173.7 | 153.3 | 11.7 | 196.3 | 96.7 | 50.8 | 44.3 | 177.3 | 139.3 | 21.4 | 175.0 | 101.0 | 42.3 | 43.0 |
| LOWER13 | 174.0 | 133.0 | 23.6 | 207.3 | 113.7 | 45.2 | 34.7 | 246.0 | 101.7 | 58.7 | 196.0 | 85.3 | 56.5 | 65.3 |
| LOWER14 | 190.3 | 142.7 | 25.0 | 183.7 | 57.7 | 68.6 | 69.7 | 267.3 | 116.0 | 56.6 | 155.3 | 76.0 | 51.1 | 71.6 |
| LOWER15 | 152.0 | 123.3 | 18.9 | 159.7 | 63.0 | 60.5 | 58.6 | 196.0 | 103.0 | 47.4 | 185.0 | 613 | 66.8 | 68.7 |
| LOWER16 | 265.7 | 91.7 | 65.5 | 250.7 | 209.7 | 16.4 | 21.1 | 261.0 | 150.0 | 42.5 | 334.7 | 154.0 | 54.0 | 41.0 |
| LOWER17 | 175.7 | 163.3 | 7.0 | 190.0 | 132.3 | 30.4 | 24.7 | 216.0 | 146.7 | 32.1 | 239.7 | 147.3 | 38.5 | 31.8 |
| LOWER18 | 153.3 | 75.0 | 51.1 | 148.3 | 94.3 | 36.4 | 38.5 | 220.0 | 95.0 | 56.8 | 146.7 | 91.0 | 38.0 | 58.6 |
| LOWER19 | 243.3 | 186.3 | 23.4 | 201.7 | 129.3 | 35.9 | 46.8 | 253.7 | 130.3 | 48.6 | 260.3 | 94.7 | 63.6 | 62.7 |
| LOWER20 | 163.7 | 111.7 | 31.8 | 184.3 | 86.3 | 53.2 | 47.3 | 213.0 | 104.7 | 50.9 | 188.3 | 89.0 | 52.7 | 58.2 |
| LOWER21 | 255.0 | 162.3 | 36.3 | 217.0 | 148.3 | 31.6 | 41.8 | 266.3 | 135.7 | 49.1 | 237.0 | 105.0 | 55.7 | 60.6 |

TABLE 2B

| | PreSup | PreSup | | PreSup | PreSup | | | PstSup | PstSup | | PstSup | PstSup | | |
|---|---|---|---|---|---|---|---|---|---|---|---|---|---|---|
| | Peak | Last | PreSup | Peak | Last | PreSup | PreSup | Peak | Last | PstSup | Peak | Last | PstSup | PstSup |
| | Torque | Torque | Set1 | Torque | Torque | Set2 | Total | Torque | Torque | Set1 | Torque | Torque | Set2 | Total |
| High Dose | Set1 | Set1 | % | Set2 | Set2 | % | % | Set1 | Set1 | % | Set 2 | Set 2 | % | % |
| (500 mg/ml) | (N-m) | (N-m) | Decline | (N-m) | (N-m) | Decline | Decline | (N-m) | (N-m) | Decline | (N-m) | (N-m) | Decline | Decline |
| UPPER1 | 158.7 | 128.7 | 18.9 | 163.7 | 151.3 | 7.5 | 4.6 | 264.7 | 141.3 | 46.6 | 172.0 | 150.7 | 12.4 | 43.1 |
| UPPER2 | 179.3 | 104.0 | 42.0 | 140.0 | 68.3 | 51.2 | 61.9 | 164.0 | 65.7 | 60.0 | 132.7 | 55.7 | 58.0 | 66.1 |
| UPPER3 | 284.7 | 209.7 | 26.3 | 287.7 | 150.3 | 47.7 | 47.2 | 297.7 | 174.3 | 41.4 | 267.7 | 134.7 | 49.7 | 54.8 |
| UPPER4 | 240.0 | 186.3 | 22.4 | 211.0 | 141.0 | 33.2 | 41.3 | 223.0 | 135.0 | 39.5 | 233.3 | 129.3 | 44.6 | 42.0 |
| UPPER5 | 189.3 | 166.0 | 12.3 | 155.7 | 126.3 | 18.8 | 33.3 | 168.0 | 98.7 | 41.3 | 148.0 | 61.7 | 58.3 | 63.3 |
| UPPER6 | 198.3 | 147.7 | 25.5 | 218.0 | 135.0 | 38.1 | 31.9 | 229.7 | 165.0 | 28.2 | 208.7 | 108.3 | 48.1 | 52.8 |
| UPPER7 | 245.7 | 84.3 | 65.7 | 171.0 | 69.7 | 59.3 | 71.6 | 219.3 | 93.7 | 57.3 | 182.0 | 65.0 | 64.3 | 70.4 |
| UPPER8 | 246.3 | 183.0 | 25.7 | 274.7 | 147.0 | 46.5 | 40.3 | 304.0 | 160.0 | 47.4 | 234.7 | 143.7 | 38.8 | 52.7 |
| UPPER9 | 201.0 | 140.7 | 30.0 | 209.7 | 152.0 | 27.5 | 24.4 | 191.3 | 163.0 | 14.8 | 280.3 | 187.7 | 33.1 | 1.9 |
| UPPER10 | 306.7 | 170.0 | 44.6 | 267.0 | 139.0 | 47.9 | 54.7 | 275.7 | 110.3 | 60.0 | 260.3 | 98.0 | 62.4 | 64.4 |
| UPPER11 | 239.3 | 123.0 | 48.6 | 203.0 | 88.3 | 56.5 | 63.1 | 230.0 | 127.3 | 44.6 | 233.3 | 108.7 | 53.4 | 52.8 |
| LOWER12 | 280.7 | 180.3 | 35.7 | 240.7 | 157.0 | 34.8 | 44.1 | 261.3 | 180.7 | 30.9 | 273.0 | 139.3 | 49.0 | 46.7 |
| LOWER13 | 154.0 | 100.0 | 35.1 | 205.3 | 128.7 | 37.3 | 16.5 | 159.0 | 106.7 | 32.9 | 146.0 | 83.0 | 43.2 | 47.8 |
| LOWER14 | 255.7 | 158.7 | 37.9 | 241.3 | 124.3 | 48.5 | 51.4 | 287.0 | 128.0 | 55.4 | 235.0 | 130.7 | 44.4 | 54.5 |
| LOWER15 | 184.0 | 129.0 | 29.9 | 195.7 | 157.3 | 19.6 | 14.5 | 234.3 | 165.7 | 29.3 | 224.3 | 197.7 | 11.9 | 15.6 |
| LOWER16 | 234.0 | 157.7 | 32.6 | 233.7 | 129.7 | 44.5 | 44.6 | 258.3 | 158.0 | 38.8 | 232.0 | 154.3 | 33.5 | 40.3 |
| LOWER17 | 227.7 | 126.7 | 44.4 | 228.0 | 98.3 | 56.9 | 56.8 | 283.3 | 144.0 | 49.2 | 258.7 | 137.0 | 47.0 | 51.6 |
| LOWER18 | 277.7 | 219.0 | 21.1 | 299.7 | 172.3 | 42.5 | 37.9 | 285.3 | 230.7 | 19.2 | 296.0 | 186.7 | 37.0 | 34.6 |
| LOWER19 | 263.0 | 162.3 | 38.3 | 217.0 | 148.3 | 31.6 | 43.6 | 241.0 | 188.7 | 21.7 | 230.3 | 135.3 | 41.2 | 43.8 |
| LOWER20 | 251.0 | 158.7 | 35.8 | 234.3 | 132.7 | 43.4 | 47.1 | 217.3 | 163.0 | 25.0 | 191.0 | 161.7 | 15.4 | 25.6 |
| LOWER21 | 251.7 | 162.0 | 35.6 | 288.7 | 123.7 | 57.2 | 50.9 | 251.0 | 150.7 | 40.0 | 247.7 | 118.7 | 52.1 | 52.7 |

TABLE2C

| | PreSup | PreSup | | PreSup | PreSup | | | PstSup | PstSup | | PstSup | PstSup | | |
|---|---|---|---|---|---|---|---|---|---|---|---|---|---|---|
| | Peak | Last | PreSup | Peak | Last | PreSup | PreSup | Peak | Last | PstSup | Peak | Last | | |
| | Torque | Torque | Set1 | Torque | Torque | Set2 | Total | Torque | Torque | Set1 | Torque | Torque | PstSup | PstSup |
| | Set1 | Set1 | % | Set2 | Set2 | % | % | Set1 | Set1 | % | Set2 | Set2 | Set2 | Total |
| Placebo | (N-m) | (N-m) | Decline | (N-m) | (N-m) | Decline | Decline | (N-m) | (N-m) | Decline | (N-m) | (N-m) | % | % |
| UPPER1 | 228.7 | 176.7 | 22.740 | 257 | 105.7 | 58.884 | 53.790 | 252.3 | 123.66 | 50.990 | 244.7 | 72.666 | 70.299 | 71.202 |
| UPPER2 | 224.0 | 193.7 | 13.541 | 212.66 | 201.7 | 5.1724 | 9.9702 | 195.3 | 175 | 10.409 | 189.0 | 156 | 17.460 | 20.136 |
| UPPER3 | 327.7 | 139.3 | 57.477 | 311.65 | 117.3 | 62.352 | 64.191 | 296.0 | 184.66 | 37.612 | 268.0 | 134.33 | 49.875 | 54.617 |
| UPPER4 | 220.3 | 174.3 | 20.877 | 269 | 149.0 | 44.609 | 32.375 | 264.3 | 193.33 | 26.860 | 279.0 | 172 | 38.351 | 34.930 |
| UPPER5 | 285.7 | 123.7 | 56.709 | 211 | 100.0 | 52.606 | 64.994 | 237.7 | 179.66 | 24.403 | 238.0 | 122.66 | 48.459 | 48.387 |
| UPPER6 | 215.0 | 144.7 | 32.713 | 241 | 128.7 | 46.611 | 40.155 | 252.3 | 157 | 37.780 | 258.7 | 144.66 | 44.072 | 42.658 |
| UPPER7 | 242.7 | 221.7 | 8.6538 | 276.66 | 196.0 | 29.156 | 19.230 | 264.7 | 182.33 | 31.108 | 295.7 | 193 | 34.723 | 27.078 |
| UPPER8 | 225.3 | 139.3 | 38.165 | 283.33 | 170.3 | 39.882 | 24.408 | 256.0 | 194 | 24.218 | 253.0 | 182.66 | 27.799 | 28.645 |
| UPPER9 | 331.3 | 208.0 | 37.223 | 330 | 169.0 | 48.787 | 48.993 | 313.0 | 152.33 | 51.331 | 277.7 | 150.66 | 45.738 | 51.863 |
| UPPER10 | 292.7 | 174.3 | 40.432 | 254 | 159.3 | 37.270 | 45.558 | 261.7 | 224 | 14.394 | 271.3 | 175.0 | 35.503 | 33.121 |
| UPPER11 | 325.7 | 157.0 | 51.791 | 283.66 | 134.3 | 52.643 | 58.751 | 298.3 | 144.33 | 51.620 | 265.0 | 112.33 | 57.610 | 62.346 |
| LOWER12 | 181.0 | 162.7 | 10.128 | 199.33 | 147.7 | 25.919 | 18.416 | 248.3 | 194 | 21.879 | 255.7 | 165.33 | 35.332 | 33.422 |
| LOWER13 | 161.3 | 125.3 | 22.314 | 188.66 | 98.0 | 48.056 | 39.256 | 268.0 | 126.66 | 52.736 | 202.0 | 122.33 | 39.438 | 54.353 |
| LOWER14 | 286.7 | 96.0 | 66.511 | 171.66 | 92.0 | 46.407 | 67.906 | 257.7 | 133 | 48.382 | 204.7 | 94.333 | 53.908 | 63.389 |
| LOWER15 | 255.0 | 109.7 | 56.993 | 228 | 101.0 | 55.701 | 60.392 | 172.3 | 152.66 | 11.411 | 198.0 | 129.33 | 34.680 | 24.951 |
| LOWER16 | 160.7 | 152.7 | 4.9792 | 244 | 129.3 | 46.994 | 19.502 | 234.0 | 119 | 49.145 | 218.0 | 97 | 55.504 | 58.547 |
| LOWER17 | 160.0 | 152.3 | 4.7916 | 163 | 120.7 | 25.971 | 24.583 | 141.3 | 111.66 | 20.990 | 145.3 | 132 | 9.1743 | 6.6037 |
| LOWER18 | 217.3 | 148.0 | 31.901 | 249.66 | 161.3 | 35.380 | 25.766 | 302.7 | 203.33 | 32.819 | 238.3 | 160.66 | 32.587 | 46.916 |
| LOWER19 | 148.7 | 134.0 | 9.8654 | 219.66 | 140.0 | 36.267 | 5.8295 | 209.3 | 112.66 | 46.178 | 218.3 | 126.33 | 42.137 | 39.649 |
| LOWER20 | 243.7 | 139.7 | 42.681 | 168.33 | 158.3 | 5.9405 | 35.020 | 208.3 | 149.66 | 28.16 | 229.7 | 135.66 | 40.928 | 34.88 |
| LOWER21 | 242.0 | 188.0 | 22.314 | 290.33 | 142.3 | 50.975 | 41.184 | 235.3 | 152 | 35.410 | 280.3 | 172.66 | 38.406 | 26.628 |

### Absolute Baseline HYP and Percent Change In HYP

Total Group (n=63; 21 Per Group)

The 3 (Group: Low, High, and Placebo)×2 (Time: Baseline and 48 hr Post)×2 (Supplementation: Pre- and Post Supplementation) mixed factorial ANOVA indicated that there was no significant (p=0.345, ηₚ²=0.035) 3-way or 2-way interaction as well as no main effects.

The 1-way ANCOVA indicated that there was no significant (p=0.122, ηₚ²=0.040) difference between groups for pre-supplementation percent decline (Baseline to 48-hrs post exercise) in HYP as well as no significant (p=0.945; ηₚ²=0.002) difference between groups for the adjusted (covaried for pre-supplementation values) mean post-supplement percent decline in HYP. An additional 1-way ANCOVA was used to evaluate the percent change in Baseline HYP pre-supplementation to Baseline HYP past-supplementation, which indicated that there was a significant (p=0.122, ηₚ²=0.105) difference between groups for pre-supplementation Baseline HYP levels, but there was no difference (p=0.252, ηₚ²=0.046) between groups for the adjusted (covaried for pre-supplementation values) mean past-supplementation Baseline HYP levels.

Upper 50^{th} Percentile (n=30; 10 Per Group)

The 3 (Group: Low, High, and Placebo)×2 (Time: Baseline and 48 hr Post)×2 (Supplementation: Pre- and Post-Supplementation) mixed factorial ANOVA indicated that there was no significant (p=0.256, ηₚ²=0.096) 3-way or 2-way interaction as well as no main effects.

The 1-way ANCOVA indicated that there was no significant (p=0.594, ηₚ²=0.079) difference between groups for pre-supplementation percent decline (Baseline to 48-hrs post exercise) in HYP as well as no significant (p=0.453; ηₚ²=0.022) difference between groups for the adjusted (covaried for pre-supplementation values) mean post-supplementation percent decline in HYP. An additional 1-way ANCOVA was used to evaluate the percent change in Baseline HYP pre-supplementation to Baseline HYP post-supplementation, which indicated that there was no significant (p=0.410, η_{hu 2}=0.026) difference between groups for pre-supplementation Baseline HYP levels, however, there was a significant difference (p=0.041, ηₚ²=0.218) between groups for the adjusted (covaried far pre-supplementation values) mean post-supplementation Baseline HYP levels. Pairwise comparisons indicated that the adjusted mean post-supplementation Baseline HYP for the high dose group (1.5±0.3 μg·mL⁻¹) was significantly (p=0.034 and p=0.024) lower than both the low dose group (2.4±0.3 μg·mL⁻¹) and placebo group (2.4±0.3 μg·mL⁻¹).

See FIG. 3; the asterisk (*) denotes the significant difference between the high dose group and the low dose and placebo groups. FIG. 3 shows ANCOVA results for the adjusted (covaried for pre-supplementation values) mean scores of post-supplementation baseline hydroxyproline. Note: Bars are X̅±Standard Error of the Mean. *High dose (500 mg·d⁻¹ of PrimaVie® Shilajit)<low dose (250 mg·d⁻¹ of PrimaVie® Shilajit; p=0.034) and placebo (p=0.024).

Lower 50^{th} Percentile (n=33; 11 Per Group)

The 3 (Group: Low, High, and Placebo)×2 (Time: Baseline and 48 hr Post)×2 (Supplementation: Pre- and Post-Supplementation) mixed factorial ANOVA indicated that there was no significant (p=0.918, ηₚ²=0.006) 3-way or 2-way interaction as well as no main effect for Group. There was, however, a significant (p=0.049, ηₚ² =0.124) main effect for Supplementation (collapsed across Group and Time). Furthermore, the pairwise comparison indicated that post-supplementation HYP levels (1.6±0.1 μg·mL⁻¹) were significantly (p=0.049, d=0.75 μg·mL⁻¹) greater than pre-supplementation (1.8±0.1 μg·mL⁻¹).

The 1-way ANCOVA indicated that there was no significant (p=0.058, ηₚ²=0.118) difference between groups for pre-supplementation percent decline (Baseline to 48-hrs post exercise) in HYP as well as no significant (p=0.760; ηₚ²=0.019) difference between groups for the adjusted (covaried for pre-supplementation values) mean post-supplementation percent decline in HYP. An additional 1-way ANCOVA was used to evaluate the percent change in Baseline HYP pre-supplementation to Baseline HYP post-supplementation, which indicated that there was no (p=0.597, ηₚ²=0.010) difference between groups for pre-supplementation Baseline HYP levels as well as no difference (p=0.540, ηₚ²=0.042) between groups for the adjusted (covaried for pre-supplementation values) mean post-supplementation Baseline HYP levels.

The 8 weeks of PrimaVie® Shilajit supplementation described above had no effects on the pre-fatigue MVIC, pre-fatigue concentric peak torque, or body weight values of the subjects. Individuals A-U designated within the groups are designated independently of study identifiers.

TABLE 3A

| | Base- | | | PostSuppl. | | |
|---|---|---|---|---|---|---|
| | line | 48-Hr | | Baseline | PostSuppl. | |
| Low Dose | HYP | (ug/ | % | HYP | 48-Hr | % |
| Group | (ug/ml) | ml) | Change | (ug/ml) | (ug/ml) | Change |
| UPPER A | 3.314 | 1.920 | −42 | 2.191 | 1.121 | −49 |
| UPPFR B | 2.482 | 2.717 | 9 | 2.573 | 2.744 | 7 |
| UPPER C | 3.301 | 2.378 | −30 | 1.890 | 2.562 | 36 |
| UPPER D | 2.399 | 1.654 | −31 | 3.278 | 2.140 | −35 |
| UPPER E | 3.409 | 2.324 | −32 | 1.910 | 1.473 | −23 |
| UPPER F | 4.747 | 2.081 | −56 | 1.693 | 1.736 | 3 |
| UPPER G | 2.609 | 3.248 | 25 | 2.425 | 2.049 | −16 |
| UPPER H | 4.300 | 3.923 | −9 | 4.229 | 2.955 | −30 |
| UPPER I | 2.497 | 2.292 | −8 | 0.979 | 2.723 | 178 |
| UPPER J | 2.001 | 3.139 | 57 | 2.620 | 2.201 | −16 |
| LOWER K | 1.084 | 0.917 | −15 | 1.501 | 2.151 | 43 |
| LOWER L | 1.388 | 1.010 | −27 | 1.389 | 0.780 | −44 |
| LOWER M | 1.432 | 1.188 | −17 | 0.581 | 1.854 | 219 |
| LOWER N | 1.928 | 1.200 | −38 | 0.948 | 1.395 | 47 |
| LOWER O | 1.669 | 1.188 | −29 | 2.247 | 1.266 | −44 |
| LOWER P | 1.788 | 1.886 | 5 | 2.084 | 1.880 | −10 |
| LOWER Q | 1.588 | 1.355 | −15 | 1.314 | 1.927 | 47 |
| LOWER R | 1.654 | 1.690 | 2 | 1.577 | 2.857 | 81 |
| LOWER S | 1.545 | 1.455 | −6 | 1.705 | 2.697 | 58 |
| LOWER T | 1.488 | 1.647 | 11 | 1.529 | 1.183 | −23 |
| LOWER U | 0.689 | 0.975 | 41 | 1.089 | 1.049 | −4 |

TABLE 3B

| | Base- | | | PostSuppl. | | |
|---|---|---|---|---|---|---|
| | line | 48-Hr | | Baseline | PostSuppl. | |
| High Dose | HYP | (ug/ | % | HYP | 48-Hr | % |
| Group | (ug/ml) | ml) | Change | (ug/ml) | (ug/ml) | Change |
| UPPER A | 4.16 | 1.64 | −61 | 1.99 | 1.35 | −32 |
| UPPER B | 3.84 | 2.18 | −43 | 0.88 | 2.08 | 136 |
| UPPER C | 3.66 | 2.21 | −40 | 2.67 | 1.56 | −42 |
| UPPER D | 3.44 | 1.79 | −48 | 1.01 | 3.30 | 227 |
| UPPER E | 2.96 | 1.67 | −44 | 1.79 | 1.42 | −21 |
| UPPER F | 2.50 | 2.91 | 16 | 1.11 | 1.54 | 39 |
| UPPER G | 2.21 | 1.37 | −38 | 1.54 | 2.28 | 49 |
| UPPER H | 2.17 | 2.74 | 26 | 1.14 | 1.51 | 33 |
| UPPER I | 1.93 | 1.25 | −35 | 0.79 | 0.83 | 5 |
| UPPER J | 1.86 | 2.43 | 30 | 2.05 | 2.12 | 4 |
| LOWER K | 1.85 | 1.06 | −43 | 1.87 | 1.76 | −6 |
| LOWER L | 1.82 | 1.76 | −4 | 1.71 | 1.76 | 3 |
| LOWER M | 1.81 | 1.87 | 4 | 2.60 | 1.76 | −32 |
| LOWER N | 1.79 | 1.27 | −29 | 3.63 | 4.28 | 18 |
| LOWER O | 1.77 | 0.94 | −47 | 1.06 | 1.27 | 20 |
| LOWER P | 1.74 | 1.29 | −26 | 1.14 | 1.42 | 25 |
| LOWER Q | 1.71 | 2.21 | 29 | 1.51 | 1.59 | 5 |
| LOWER R | 1.69 | 1.67 | −1 | 1.51 | 1.04 | −31 |
| LOWER S | 1.59 | 0.93 | −42 | 1.30 | 2.06 | 58 |
| LOWER T | 1.52 | 1.81 | 20 | 1.37 | 1.13 | −18 |
| LOWER U | 1.38 | 2.04 | 48 | 2.20 | 3.62 | 64 |

TABLE 3C

| | Base- | | | PostSuppl. | PostSuppl. | |
|---|---|---|---|---|---|---|
| | line | 48-Hr | | Baseline | 48-Hr | |
| Placebo | HYP | (ug/ | % | HYP | HYP | % |
| Group | (ug/ml) | ml) | Change | (ug/ml) | (ug/ml) | Change |
| UPPER A | 4.80 | 3.46 | −28 | 3.02 | 1.55 | −49 |
| UPPER B | 3.38 | 2.07 | −39 | 2.23 | 4.90 | 120 |
| UPPER C | 3.18 | 3.28 | 3 | 3.39 | 2.97 | −13 |
| UPPER D | 3.02 | 1.83 | −39 | 1.10 | 0.85 | −23 |
| UPPER E | 2.49 | 2.37 | −5 | 1.13 | 2.01 | 77 |
| UPPER F | 2.37 | 2.18 | −8 | 2.07 | 1.38 | −33 |
| UPPER G | 2.35 | 1.74 | −26 | 3.73 | 1.96 | −48 |
| UPPER H | 2.31 | 1.96 | −15 | 2.00 | 1.96 | −2 |
| UPPER I | 2.21 | 3.80 | 72 | 3.40 | 1.57 | −54 |
| UPPER J | 1.93 | 2.73 | 41 | 1.78 | 2.07 | 16 |
| LOWER K | 1.87 | 1.77 | −6 | 2.17 | 2.70 | 24 |
| LOWER L | 1.86 | 2.12 | 14 | 1.47 | 2.31 | 57 |
| LOWER M | 1.84 | 2.03 | 10 | 1.64 | 1.79 | 9 |
| LOWER N | 1.58 | 2.86 | 80 | 0.97 | 1.91 | 96 |
| LOWER O | 1.52 | 3.26 | 114 | 1.42 | 6.81 | 379 |
| LOWER P | 1.48 | 3.44 | 132 | 1.58 | 1.98 | 26 |
| LOWER Q | 1.39 | 1.63 | 18 | 1.23 | 1.33 | 8 |
| LOWER R | 1.34 | 1.32 | −1 | 3.01 | 3.12 | 4 |
| LOWER S | 1.26 | 1.47 | 16 | 1.63 | 2.05 | 25 |
| LOWER T | 1.17 | 0.98 | −16 | 0.94 | 0.94 | −0.3 |
| LOWER U | 1.08 | 1.56 | 43 | 2.32 | 2.40 | 4 |

Table 4 provides the X̅±SD values for the total group and subgroups for each dependent variable in the present study. There was a significant (p=0.005, ηₚ²=0.122) main effect for Fatigue (collapsed across Group and Visit), and the pairwise comparison indicated that Pretest MVIC (341.8±8.3 Nm) was significantly (p=0.005; d=0.27) greater than Posttest MVIC (323.±8.3 Nm).

The 1-way ANCOVA and pairwise comparisons indicated that the adjusted mean post-supplementation percent decline in MVJC was significantly (p=0,021; d=0.42) less for the high dose (9.1±2.0%) than the low dose (15.7±2.0%).

TABLE 4
Descriptive Characteristics (X̅ ± SD)

| | Pre-Supplementation | | | Post-Supplementation | | |
|---|---|---|---|---|---|---|
| | Total | Upper | Lower | Total | Upper | Lower |
| MVIC (Nm) | | | | | | |
| Low Dose | 376.1 ± 79.1 | 447.3 ± 45.8 | 311.5 ± 30.9 | 360.6 ± 95.3 | 431.9 ± 54.2 | 295.8 ± 76.3 |
| High Dose | 366.0 ± 53.5 | 409.8 ± 44.2 | 326.3 ± 18.0 | 335.3 ± 53.0 | 351.6 ± 59.7 | 320.5 ± 43.7 |
| Placebo | 366.3 ± 81.8 | 431.7 ± 60.5 | 306.8 ± 43.8 | 346.8 ± 63.6 | 370.4 ± 50.8 | 325.3 ± 68.5 |
| % Decline MVIC | | | | | | |
| Low Dose | 16.5 ± 9.5 | 19.8 ± 9.1 | 14.1 ± 9.5 | 16.0 ± 12.2 | 18.2 ± 9.8 | 14.0 ± 14.1 |
| High Dose | 14.1 ± 9.4 | 16.6 ± 10.8 | 11.8 ± 7.6 | 9.0 ± 6.9 | 9.0 ± 7.5 | 9.1 ± 6.6 |
| Placebo | 13.0 ± 10.1 | 12.4 ± 10.0 | 14.2 ± 11.1 | 14.2 ± 6.9 | 14.8 ± 6.2 | 13.8 ± 7.8 |
| Peak Torque (Nm) | | | | | | |
| Low Dose | 224.5 ± 49.0 | 267.4 ± 24.0 | 185.6 ± 28.0 | 253.0 ± 48.8 | 274.2 ± 28.6 | 233.7 ± 56.3 |
| High Dose | 231.8 ± 42.5 | 266.3 ± 20.3 | 200.5 ± 31.1 | 240.2 ± 43.4 | 264.0 ± 31.2 | 218.7 ± 42.7 |
| Placebo | 236.9 ± 56.3 | 283.3 ± 36.4 | 194.8 ± 32.1 | 246.1 ± 42.9 | 254.5 ± 43.2 | 238.5 ± 43.2 |
| % Decline Peak Torque | | | | | | |
| Low Dose | 44.6 ± 15.1 | 46.9 ± 16.6 | 42.5 ± 13.8 | 51.5 ± 15.5 | 49.3 ± 15.7 | 53.4 ± 15.9 |
| High Dose | 42.0 ± 16.8 | 41.2 ± 19.4 | 42.7 ± 15.0 | 46.5 ± 16.5 | 50.0 ± 13.2 | 43.4 ± 19.2 |
| Placebo | 38.1 ± 18.9 | 42.2 ± 19.6 | 34.4 ± 18.3 | 41.2 ± 16.4 | 50.6 ± 15.6 | 39.7 ± 18.1 |
| HYP (ug/mL) | | | | | | |
| Low Dose | 2.25 ± 1.1 | 3.11 ± 0.9 | 1.48 ± 0.3 | 1.89 ± 0.8 | 2.38 ± 0.9 | 1.45 ± 0.5 |
| High Dose | 2.26 ± 0.8 | 2.87 ± 0.9 | 1.70 ± 0.1 | 1.66 ± 0.7 | 1.50 ± 0.6 | 1.81 ± 0.8 |
| Placebo | 2.12 ± 0.9 | 2.80 ± 0.8 | 1.50 ± 0.3 | 2.02 ± 0.9 | 2.40 ± 0.9 | 1.68 ± 0.6 |
| % Change HYP | | | | | | |
| Low Dose | 9.63 ± 27.8 | 11.5 ± 34.0 | 7.2 ± 23.3 | 20.3 ± 70.2 | 5.5 ± 65.2 | 32.9 ± 78.9 |
| High Dose | 15.6 ± 32.9 | 23.6 ± 33.8 | 4.8 ± 31.3 | 24.0 ± 62.1 | 39.8 ± 83.1 | 11.2 ± 33.2 |
| Placebo | 17.2 ± 47.7 | 4.3 ± 35.7 | 41.1 ± 50.7 | 29.7 ± 92.0 | 0.81 ± 57.6 | 60.7 ± 115.7 |

### Discussion

The above Example shows that the administration of 500 mg·d⁻¹ of PrimaVie® Shilajit to subjects for 8 weeks reduced muscle fatigue, increasing retention of maximal muscular strength following a fatiguing task and decreasing baseline HYP in the upper 50^{th} percentile of subjects.

Specifically, for the total group (n=63; 21 per group), the percent decline in MVIC following the fatiguing protocol was less for the 500 mg·d⁻¹ of PrimaVie® Shilajit group than the 250 mg·d⁻¹ group. In addition, the examination of the sub-sample of subjects in the upper 50^{th} percentile of MVIC indicated that the high dose group was more resistant to fatigue and retained a greater level of maximal muscular strength than both the low dose and placebo groups.

The above data shows the beneficial effects of PrimaVie° Shilajit supplementation on fatigue-related performance outcomes using a resistance exercise model. Previous studies, however, have examined the effects of PrimaVie° Shilajit supplementation on mitochondrial indices of exercise performance that may have implications for the findings of the present study. For instance, Bhattacharyya et al. (Pharmacologyonline 2009; 1:817-25) used a forced swimming task in mice to examine the effects of PrimaVie® Shilajit supplementation on indices of mitochondrial function including post-exercise muscle ATP concentration and adenylate energy charge. Oral PrimaVie® Shilajit supplementation (30 mg·kg⁻¹ of body weight for 4 days) resulted in a significantly greater (p<0.001) post-exercise ATP concentration of 0.49±0.05 μmol·g⁻¹ of muscle compared to 0.25±0.05 μmol·g⁻¹ for the swimming only group without PrimaVie® Shilajit supplementation. Furthermore, the adenylate energy charge, which is an index of the energy status of the cell and depends on the relative concentrations of ATP, adenosine diphosphate, and adenosine monophosphate, was significantly (p<0.05) greater post-exercise for the PrimaVie® Shilajit supplementation plus swimming group (0.62±0.06 units) compared to the swimming only without PrimaVie® Shilajit supplementation (0.52±0.04 units). Bhattacharyya et al. (2009) hypothesized that the augmented mitochondrial function, improved energy status, and upregulated ATP synthesis were the result of PrimaVie® Shilajit supplementation due to “. . . its potent electron transfer capacity and antioxidant activity” (p. 823).

Without being bound by theory, the enhanced mitochondrial function reported by Bhattacharyya et al. (2009) may at least in part explain the improved resistance to fatigue found in the present study for the 500 mg·d⁻¹ PrimaVie® Shilajit group. Krustrup et al. (“ATP and heat production in human skeletal muscle during dynamic exercise: higher efficiency of anaerobic than aerobic ATP resynthesis” J Physiol 2003; 549(1):255-69) demonstrated that mitochondrial respiration contributes to the resynthesis of ATP during high intensity leg extension muscle actions similar to those used during the fatiguing protocols in the present study. Furthermore, given the beneficial findings for the upper 50^{th} percentile of MVIC group in the present study, this explanation may be particularly applicable to an athlete and/or other subject involved in resistance training. Improved mitochondrial functioning may also explain the findings of a recent unpublished, pilot study of six adult humans that found improved performance on the Harvard Step Test following 15 days of supplementation with 200 mg of PrimaVie® Shilajit.

In one embodiment of this invention, Shilajit (such as PrimaVie® Shilajit) in conjunction with resistance training enhances strength gains as well as increases in muscle mass and body Weight due to Shilajit's effect on circulating testosterone levels. PrimaVie® Shilajit plus resistance training increases muscular strength, size, and endurance. See for instance Pandit et al, (Andrologia 2016; 48(5):570-5), reporting that 500 mg·d⁻¹ PrimaVie® Shilajit for 90 days increased baseline testosterone (4.84±1.54 ng·mL⁻¹), free testosterone (15.36±7.17 pg·mL⁻¹), and dehydroepiandrosterone (145.09±53.17 μg·dL⁻¹) by 20.45%, 19.14%, and 31.35%, respectively in healthy adult men. in an embodiment, a method of the present invention includes the administration of PrimaVie® Shilajit along with resistance training to synergistically increase muscular strength, size, and appearance.

In the above Example, 8 weeks of PrimaVie® Shilajit supplementation at 500 mg·d⁻¹ resulted in a 29% post-supplementation reduction in baseline hydroxyproline (HYP) in the sub-sample of subjects in the upper 50^{th} percentile for pre-supplementation HYP. The reduced baseline HYP shows a reduction in collagen degradation following supplementation. Previous studies (Suominen et al., “Effects of physical training on metabolism of connective tissues in young mice” Acta Physiol Scand 1980; 108(1):17-22; Tofas et al., “Plyometric exercise increases serum indices of muscle damage and collagen breakdown” J Strength Cond Res 2008; 22(4490-6; Brown et al., “Indices of skeletal muscle damage and connective tissue breakdown following eccentric muscle contractions” Eur J Appl Physiol Occup Physiol 1997; 75(4369-74; Kovanen et al., “Collagen of slow twitch and fast twitch muscle fibres in different types of rat skeletal muscle” Eur J Appl Physiol Occup Physiol 1984; 52(4235-42) have used HYP as an indirect indicator of collagen degradation from muscle and connective tissues. Exercise-induced muscle damage and soreness (Clarkson et al., “Exercise-induced muscle damage in humans” Am J Phys Med Rehabil 2002; 81(11):S52-69), connective tissue disruption (Saxton et al., “Neuromuscular dysfunction following eccentric exercise” Med Sci Sports Exerc” 1995; 27(8):1185-93), and elevated HYP (Brown et al., “Indices of skeletal muscle damage and connective tissue breakdown following eccentric muscle contractions” Eur J Appl Physiol Occup Physiol 1997; 75(4):369-74; Brown et al., “Indirect evidence of human skeletal muscle damage and collagen breakdown after eccentric muscle actions” J Sports Sci 1999; 17(5):397-402) often result from eccentric, but not concentric, muscle actions. The lack of acute (baseline versus 48 hr post-exercise) changes in HYP in the present study following the pre-supplementation and post-supplementation fatiguing work bouts were likely due to the use of concentric muscle actions, which typically cause little or no muscle damage (Clarkson et al., “Exercise-induced muscle damage in humans” Am J Phys Med Rehabil 2002; 81(11):S52-69; Byrne et al., “Neuromuscular function after exercise-induced muscle damage” Sport Med 2004 Jan. 1; 34(1):49-69). In one embodiment, the post-supplementation reduction in baseline HYP was due to a reduction in collagen degradation associated with connective tissues, but not muscle, showing that 8 weeks of PrimaVie® Shilajit supplementation at 500 mg·d⁻¹ supported connective tissue health associated with tendons and ligaments. See also Baar et al., “Minimizing injury and maximizing return to play: Lessons from engineered ligaments” Sport Med 2017; 47(45-11.

In one embodiment, a subject of the present invention is a competitive and/or recreational athlete involved in high volumes of chronic exercise who exhibits elevated exercise-induced collagen degradation. Das et al. (J Med Food 2016; 19(7):701-9) found that 8 weeks of PrimaVie® Shilajit supplementation at 500 mg·d⁻¹ increased mRNA expression of collagen, the major structural protein in the skeletal muscle extracellular matrix, which accounts for 6% of the weight of tendinous muscle. See also for instance Gisselman et al., “Musculoskeletal overuse injuries and heart rate variability: Is there a link?” Med Hypotheses 2016; 87:1-7; Magnusson et al., “The impact of loading, unloading, ageing and injury on the human tendon” J Physiol 2018; 597(5):1283-1298; Perry, “Exercise, injury and chronic inflammatory lesions” British Medical Bulletin 1992; 1; 48(3):668-82).

In an embodiment, a subject of a method of the present invention has impaired collagen function, such as subject having Padget's disease, Ehlers-Danos Syndrome, Osteogenesis Imperfecta, and/or another collagen-related disorder.

In an embodiment, a subject of a method of the present invention is an athlete whose exercise conditions cause muscle damage such as high intensity eccentric muscle actions in healthy individuals.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. A method of reducing collagen degradation in a subject comprising the steps of:
(a) providing a composition comprising Shilajit, or its individual components, or a combination of at least (i) and (ii) below, the Shilajit or individual components or combination comprising (i) at least 10,3% by weight combined of free dibenzo-α pyrones and dibenzo-α-pyrones conjugated with chromoproteins and (ii) at least 50% by weight of fulvic acids with dibenzo-α-pyrone core nucleus;
(b) administering the composition to the subject to deliver the Shilajit or its individual components or combination to the subject's bloodstream and bodily tissues in an amount effective to reduce collagen degradation in the tissues of the subject; and
(c) said Shilajit or components or combination thereof acting on said tissues to reduce collagen degradation therein.

2. The method of claim 1, wherein the Shilajit, individual components, or combination thereof in the composition is a standardized aqueous Shilajit extract.

3. The method of claim 2, wherein the standardized aqueous Shilajit extract is in dry powder form with 14-17% (w/w) combined of free dibenzo-a pyrones and dibenzo-α-pyrones conjugated with chromoproteins, and 59-62% (w/w) fulvic acids with dibenzo-α pyrone core nucleus.

4. The method of claim 2, wherein said composition is a dietary supplement and said administering step (b) is by oral administration of the dietary supplement.

5. The method of claim 4, wherein said effective amount of step (b) is about 50 mg to about 1500 mg of standardized aqueous Shilajit extract per day, and said subject is human.

6. The method of claim 4, wherein said effective amount of step (b) is 200 mg to 500 mg of standardized aqueous Shilajit extract per day, and said subject is human.

7. The method of claim 5, wherein said dietary supplement is administered daily for at least 8 weeks. 8, The method of claim 6, wherein said dietary supplement is administered daily for at least 8 weeks. 9, The method of claim 4, wherein said effective amount is about 1 to about 22 mg of the standardized aqueous Shilajit extract, per kilogram of the subject, per day.

10. The method of claim 9, wherein said effective amount is about 5 mg/kg to about 10 mg/kg.

11. The method of claim 10, wherein said dietary supplement is administered daily for at least 8 weeks.

12. The method of claim 5, wherein said subject is a human adult athlete having a baseline, pre-administration serum hydroxyproline concentration of at least 0.7 ug/ml.

13. The method of claim 12, said subject having a baseline, pre-administration serum hydroxyproline concentration of at least 2.0 ug/mL.

14. The method of claim 13, step (c) further comprising reducing serum hydroxyproline concentrations In the subject by 10-77%.

15. The method of claim 14, step (c) further comprising reducing serum hydroxyproline concentrations in the subject by at least 29%.

16. The method of claim 14, step (b) further comprising administering the standardized aqueous Shllajit extract daily for at least 8 weeks.

17. The method of claim 14, further comprising step (d) performing resistance training to increase the muscle strength and/or mass, and/or body weight of the subject.

18. The method of claim 5, wherein collagen degradation in step (c) is reduced in connective tissue of the subject, and said method supports and/or improves connective tissue health in the subject.

19. The method of claim 5, wherein collagen degradation in step (c) is reduced in tendons and/or ligaments of the subject, and said method supports and/or improves tendon and ligament health in the subject.

20. The method of claim 19, said method improving skeletal muscle strength, muscle building and muscle generation in the subject.

21. The method of claim 5, wherein collagen degradation in step (c) is reduced in the tissues of the subject, and said method supports and/or improves the health of said tissue or tissues.

22. The method of claim 5, wherein collagen degradation in step (c) is reduced in the subject's skeletal muscle tissue and/or associated connective tissue, said method further comprising the step of, after step (c), reducing muscle fatigue in the subject under or after fatiguing conditions.

23. The method of claim 22, wherein said effective amount is 100-1000 mg of the standardized aqueous Shilajit extract per day.

24. The method of claim 22, wherein collagen degradation in step (c) is reduced in the subject's skeletal muscle tissue and/or associated connective tissue, said method further comprising the step of, after step (c), increasing skeletal muscle strength in the subject.

25. The method of claim 24, further comprising step (d), performing resistance training to increase the muscle strength and/or mass, and/or body weight of the subject.

26. The method of claim 22, wherein said muscle fatigue is measured by a decline in Maximal Voluntary Isometric Contraction strength in the subject under or after fatiguing conditions, and the subject's Maximal Voluntary Isometric Contraction strength decline is reduced by at least 6% after administration of the Shilajit extract, as compared with pre-administration muscle strength decline.

27. The method of claim 26, wherein said decline in Maximal Voluntary Isometric Contraction strength is reduced by 6 to 25% after administration of the Shilajit extract.

28. The method of claim 5, wherein said subject has osteoporosis.

29. The method of claim 5, wherein said subject has arthritis.

30. A method of reducing muscle fatigue in a subject comprising the steps of:
(a) providing a composition comprising Shilajit, or its individual components, or a combination of at feast (i) and (ii) below, the Shilajit or individual components or combination comprising (i) at least 10.3% combined by weight of free dibenzo-α-pyrones and dibenzo-α-pyrones conjugated with chromoproteins, and (ii) at least 50% by weight of fulvic acids with dibenzo-α-pyrone core nucleus;
(b) administering an effective amount of the Shilajit or individual components or combination to the subject to deliver the Shilajit or individual components or combination to the subject's bloodstream and skeletal muscle tissue and/or associated connective tissue to at least reduce collagen degradation;
(c) said Shilajit or components or combination thereof acting on the subject's skeletal muscle tissue and/or associated connective tissue to reduce collagen degeneration in said tissue or tissues;
(d) introducing fatiguing conditions to the subject's skeletal muscle tissue; and
(e) said skeletal muscle tissue retaining muscle strength under the fatiguing conditions and reducing muscle fatigue.

31. The method of claim 30, wherein the Shilajit, individual components, or combination in the composition is a standardized aqueous Shilajit extract.

32. The method of claim 31, wherein the standardized aqueous Shilajit extract is in dry powder form with 1447% (w/w) combined of free dibenzo-a pyrones and dibenzo-α-pyrones conjugated with chromoproteins, and 59-62% (w/w) fulvic acids with dibenzo-α pyrone core nucleus.

33. The method of claim 31, wherein said composition is a dietary supplement and said administering step is by oral administration of the dietary supplement, daily.

34. The method of claim 33, wherein said subject is human and said effective amount is about 50 to about 1500 mg per day of the Shilajit.

35. The method of claim 33, wherein said subject is human and said effective amount is about 200 mg to about 500 mg per day of the Shilajit.

36. The method of claim 34, wherein said dietary supplement is administered daily for at least 8 weeks.

37. The method of claim 33, wherein said effective amount is about 1 to about 22 mg of the standardized Shilajit per kilogram of the subject.

38. The method of claim 37, wherein said effective amount is about 5 mg/kg to about 10 mg/kg.

39. The method of claim 38, wherein said dietary supplement is administered daily for at least 8 weeks.

40. The method of claim 34, wherein the subject is human and an adult athlete having a baseline, pre-administration serum hydroxyproline concentration of at least 0.7 ug/ml.

41. The method of claim 40, said subject having a baseline, pre-administration serum hydroxyproline concentration of at least 2.0 ug/ml.

42. The method of claim 41, step (c) further comprising reducing serum hydroxyproline concentration in the subject by 10-77%,

43. The method of claim 42, step (c) further comprising reducing serum hydroxyproline concentration in the subject by at least 29%.

44. The method of claim 41, further comprising the step of said athlete performing resistance training to increase the muscle strength and/or mass, and/or body weight of the subject.

45. The method of claim 34, said method improving the retention of muscle strength during or after fatiguing conditions.

46. The method of claim 34, wherein said muscle fatigue is measured by a decline in Maximal Voluntary Isometric Contraction strength in the subject under or after fatiguing conditions, and the subject's Maximal Voluntary Isometric Contraction strength decline is reduced by at least 6% after administration of the Shilajit extract, as compared with pre-administration muscle strength decline.

47. The method of claim 46, wherein said decline in Maximal Voluntary Isometric Contraction strength is reduced by 6 to 25% after administration of the Shilajit extract.

48. The method of claim 31, wherein said Shilajit or individual components or combination includes at least 60% fulvic acids with dibenzo-at-pyrone core nucleus.

49. A method of improving skeletal muscle tissue and/or connective tissue in a subject comprising the steps of:
(a) providing a composition comprising Shilajit, or its individual components, or a combination of at least (i) and (ii) below, the Shilajit or individual components or combination comprising (i) at least 10.3% combined by weight of free dibenzo-α-pyrones and dibenzo-α-pyrones conjugated with chromoproteins, and (ii) at least 50% by weight of fulvic acids with dibenzo-α-pyrone core nucleus;
(b) administering an effective amount of the Shilajit or individual components or combination to the subject to deliver the Shilajit or individual components or combination to the subject's bloodstream and skeletal muscle tissue and/or associated connective tissue at least to reduce collagen degradation in those tissues; and
(c) said Shilajit or components or combination thereof acting on said skeletal muscle tissue and/or associated connective tissue to reduce collagen degradation and thereby improve the skeletal muscle and/or associated connective tissue.

50. The method of claim 49, wherein the Shilajit, individual components, or combination thereof in the composition is a standardized aqueous Shilajit extract.

51. The method of claim 50, wherein said composition is a dietary supplement and said administering step (b) is by oral administration of the dietary supplement.

52. The method of claim 51, wherein said effective amount of step (b) is about 50 mg to about 1500 mg of standardized aqueous Shilajit extract per day, and said subject is human.

53. The method of claim 52, wherein said effective amount of step (b) is 200 mg to 500 mg of standardized aqueous Shilajit extract per day, and said subject is human.

54. The method of claim 51, wherein said effective amount is about 1 to about 22 mg of the standardized aqueous Shilajit extract, per kilogram of the subject per day.

55. The method of claim 52, wherein said dietary supplement is administered daily for at least 8 weeks.

56. The method of claim 52, further comprising the step of performing resistance training to increase muscular strength and/or mass, and/or the body weight of the subject,

57. A method of treating osteoporosis or arthritis in a subject, comprising the steps of
(a) providing a composition comprising Shilajit, or its individual components, or a combination of at least (i) and (ii) below, the Shilajit or individual components or combination comprising (i) at least 10.3% by weight combined of free dibenzo-α pyrones and dibenzo-α-pyrones conjugated with chromoproteins and (ii) at least 50% by weight of fulvic acids with dibenzo-α-pyrone core nucleus;
(b) administering the composition to the bloodstream of the subject to deliver the Shilajit or its individual components or combination to the subject's bloodstream and bodily tissues in an amount effective to reduce collagen degradation in the tissues of the subject; and
(c) said Shilajit or components or combination thereof acting on said tissues to reduce collagen degradation therein.

58. The method of claim 57, wherein the Shilajit, individual components, or combination thereof in the composition is a standardized aqueous Shilajit extract.
